(19) United States
(12) Patent Application Publication
(10) Pub. No.: US 2003/0091579 A1
(43) Pub. Date: May 15, 2003

(54) METHODS OF RAISING ANIMALS FOR MEAT PRODUCTION

(76) Inventors: JACK G. MANNS, SASKATOON (CA); STEPHEN D. ACRES, SASKATOON (CA); RICHARD HARLAND, SASKATOON (CA)

Correspondence Address: Ronald L. Stotish, Vice President, Research & Development, MetaMorphix International, Inc., 8510A Corridor Road, Savage, MD, 20763, US

(21) Appl. No.: 09/305,924

(22) Filed: May 5, 1999

Related U.S. Application Data
(60) Provisional application No. 60/084,217, filed on May 5, 1998.

(51) Int. Cl. A61K 39/00; A61K 39/395

(52) U.S. Cl. 424/185.1; 424/145.1

(57) ABSTRACT

Methods for raising uncastrated male animals for meat production are disclosed. The methods use compositions which include GnRH immunogens. The methods are useful for producing cuts of meat with enhanced organoleptic qualities.

### CROSS-REFERENCE TO RELATED APPLICATION

This application is related to provisional patent application serial No. 60/084,217, filed May 5, 1998, from which priority is claimed under 35 USC §119(e) (1) and which is incorporated herein by reference in its entirety.

### TECHNICAL FIELD

The present invention relates generally to methods for raising animals for meat production. More particularly, the invention is directed to methods of immunizing animals with a primary vaccination of a GnRH immunogen which causes a reduction in circulating gonadal steroid levels, followed by revaccination with a GnRH immunogen shortly before slaughter to substantially reduce the level of one or more androgenic and/or non-androgenic steroids.

### BACKGROUND OF THE INVENTION

Male cattle, pigs, and sheep are typically more heavily muscled and have a larger mature body size than females. The conventional explanation is that as male animals reach sexual maturity, the secretion of testosterone, the anabolic steroid produced by the testes, results in increased muscle protein deposition and decreased fat. Numerous studies have also established that males utilize feed more efficiently than females during the growing period (Field, R. A., J. Animal Sci. (1971) 32:849-858). These differences are most obvious around the time of puberty and testosterone has an important role in regulating these changes.

In males, androgenic steroids, testosterone and androsterone, are involved in regulating two different biological processes. One effect is physiologic and results in increased muscle deposition, reduced fat synthesis, and increased efficiency of feed utilization. Testosterone has similar direct effects on growth of sex glands such as seminal vesicles, the prostate gland and testes. These actions involve direct interaction of the androgen with receptors in target tissues. The second general effect of androgens is to cause sexual and other behavioral changes typical of males. Those effects are mediated through the central nervous system. It is possible that because these various effects occur in different tissues by different molecular mechanisms, some of them may be maintained at low plasma concentrations of androgen whereas other effects may require higher levels.

Although it is generally assumed that androgens are required early in life to maintain optimal growth, muscle deposition and feed efficiency, that assumption may not be true even though measurable amounts of androgen are present. Alternatively, androgen-sensitive tissues, such as muscle, may be much more responsive to lower levels of androgen early in life than at puberty or later. Allrich et al., J. Animal Sci. (1982) 55:1139-1146, compared rates of gain in body weight and weights of testosterone-sensitive tissues in pigs as a function of age and testosterone concentration.

In particular, a comparison of the rate of growth of different tissues at different ages showed that tissue sensitivity to testosterone, as well as testosterone concentration, changed with age. For example, the authors showed that from day 40 to 100, while serum testosterone increased 3.1 fold, body weight increased 3.2 fold, testes weight 3.6 fold and seminal vesicles 4.0 fold. These data also showed that prepubertal testosterone levels were low but readily detectable, and that body weight increased at approximately the same rate as the other tissues in the presence of low levels of testosterone. Pigs begin to reach sexual maturity at approximately 130 to 150 days of age (65 to 85 kg body weight) and serum testosterone concentrations increase significantly at that time. From day 100 to 190, while serum testosterone increased 4.0 fold, body weight, testicular weight, and seminal vesicle weight increased 2.6 fold, 13.5 fold, and 9.2 fold, respectively.

Furthermore, Knudson et al., J. Animal Sci. (1985) 61:789-796, showed that castrated male pigs gained weight at a similar rate compared to intact males until about 90 days of age, but beyond that age intact males grew more efficiently than castrates. This feature is particularly important for immunosterilization of herd animals, and particularly where it is desired to immunocastrate male piglets to prevent “boar taint” which is produced by the synthesis of sex steroids in normally functioning testicles of male piglets. See e.g. Meloen et al., Vaccine (1994) 12(8):741-746.

A large number of studies have been done in pigs and cattle to explore the use of GnRH immunization as a method of improving growth rate and feed efficiency in animals. See, e.g, Adams and Adams, J. Animal Sci. (1992) 70:1691-1698; Caraty and Bonneau, C.R. Acad. Sc. Paris (1986) 303:673-676; Chaffaux et al., Recueil de Medecine Veterinaire (1985) 161:133-145; Finnerty et al., J. Repro. Fertil. (1994) 101:333-343. The objective of many of these studies has been to allow animals to grow as intact males until the approach of the end of the fattening period and then to immunologically castrate them. To achieve immunological castration towards the end of the fattening period and just prior to slaughter, the animals are vaccinated one or more times earlier in life to prime the immune system so it will respond strongly to the revaccination given towards the end of the fattening period. The first vaccination is designed to prime the immune system to the GnRH antigen but to avoid inducing high anti-GnRH antibody titers which would reduce serum testosterone levels or prevent it from increasing as animals approach puberty. This was based on the belief that reducing serum testosterone would also reduce growth rate or feed efficiency in young animals.

For example, Meloen et al., Vaccine (1994) 12:741-746, describe the use of a GnRH tandem vaccine, administered in two doses, in order to reduce boar taint in pigs. No reduction in testicle size occurred until after the second immunization. See, also, International Publication No. WO 90/11298, published Oct. 4, 1990. Falvo et al., J. Anim. Sci. (1986) 63:986-994 report the use of GnRH vaccines to study various effects in pigs, including the presence of boar taint and carcass characteristics. The authors report that plasma testosterone levels were significantly reduced two weeks following the first booster injection. Similarly, U.S. Pat. No. 5,573,767, pertains to a method of improving the organoleptic qualities of meat using GnRH immunization. The method entails two immunizations, one immunization designed to have no effect on gonadal steroid secretion and a second immunization before slaughter in order to abolish the action of androgenic and non-androgenic steroids.

Additionally, prior attempts at immunosterilization have not produced uniform results due to the insufficient immunogenicity of GnRH peptides and/or related carrier systems, and the resultant inability of various prior GnRH-based vaccines to induce sufficient immune responses toward endogenous GnRH. See, e.g., Robertson, Vet. Record (1981) 108:381-382. Accordingly, reliable methods for immunosterilization of food-producing animals would be desirable.

### DISCLOSURE OF THE INVENTION

The present invention is based on a reliable, reproducible method for raising a food-producing male animal for meat production. In particular, contrary to the prevailing belief, it appears, based on the trials described herein, that avoiding a substantial reduction in testosterone early in life is not necessary in order to produce commercially acceptable quantities of meat, and that primary GnRH immunization that induces antibodies that have a measurable effect on gonadal steroid secretion during the fattening period can be achieved without significant loss of growth rate or feed efficiency. The primary immunization can be followed later in life with a secondary immunization that abolishes the action of androgenic and/or non-androgenic steroids.

Accordingly, in one embodiment, the invention is directed to a method of raising an uncastrated male food-producing animal for meat production comprising vaccinating the animal with a first vaccine composition comprising a GnRH immunogen prior to or during the fattening period of the animal to cause a reduction in circulating testosterone levels, and vaccinating the animal with a second vaccine composition comprising a GnRH immunogen at about 2 to about 8 weeks before slaughter of the animal to substantially reduce the level of one or more androgenic and/or non-androgenic steroids.

In particularly preferred embodiments, the first and/or second vaccine compositions comprise an immunological adjuvant such as an adjuvant comprising an oil and dimethyldioctadecylammonium bromide. Furthermore, the GnRH immunogen in the first and/or second vaccine composition may be a GnRH multimer comprising the general formula (GnRH-X-GnRH)y wherein:

GnRH is a GnRH immunogen;

X is one or more molecules selected from the group consisting of a peptide linkage, an amino acid spacer group, a carrier molecule and [GnRH]ₙ, where n is an integer greater than or equal to 1; and

y is an integer greater than or equal to 1.

In certain embodiments, administration of the first vaccine composition results in the production of antibodies that cross-react with endogenous GnRH of the animal and the second composition is administered after the antibody levels have declined.

In another embodiment, the invention is directed to a method of raising an uncastrated male bovine, ovine or porcine animal for meat production comprising vaccinating the animal with a first vaccine composition comprising a GnRH immunogen prior to or during the fattening period of said animal to cause a reduction in circulating testosterone levels, and vaccinating the animal with a second vaccine composition comprising a GnRH immunogen at about 2 to about 8 weeks before slaughter of the animal, to substantially reduce the level of one or more androgenic and/or non-androgenic steroids. The first and/or second vaccine compositions may further comprise an immunological adjuvant.

In yet another embodiment, the invention is directed to a method of raising an uncastrated male bovine, ovine or porcine animal for meat production comprising:

(a) vaccinating the animal with a first vaccine composition comprising an immunological adjuvant and a GnRH multimer comprising the general formula (GnRH-X-GnRH)y wherein:

GnRH is a GnRH immunogen;

X is one or more molecules selected from the group consisting of a peptide linkage, an amino acid spacer group, a leukotoxin polypeptide and [GnRH]ₙ, where n is an integer greater than or equal to 1; and

y is an integer greater than or equal to 1, wherein said first vaccine composition is administered prior to or during the fattening period of the animal to cause a reduction in circulating testosterone levels; and

(b) vaccinating the animal with a second vaccine composition comprising an immunological adjuvant and a GnRH multimer comprising the general formula (GnRH-X-GnRH)y wherein:

GnRH is a GnRH immunogen;

X is one or more molecules selected from the group consisting of a peptide linkage, an amino acid spacer group, a leukotoxin polypeptide and [GnRH]ₙ, where n is an integer greater than or equal to 1; and

y is an integer greater than or equal to 1, wherein said second vaccine composition is administered at about 2 to about 8 weeks before slaughter of the animal, to substantially reduce the level of one or more androgenic and/or non-androgenic steroids.

In yet another embodiment, the invention is directed to a method of raising an uncastrated male bovine, ovine or porcine animal for meat production comprising:

(a) vaccinating the animal with a first vaccine composition comprising an immunological adjuvant and a GnRH multimer comprising the amino acid sequence depicted in FIGS. 3A-3F (SEQ ID NO:______), or an amino acid sequence with at least about 75% sequence identity thereto, wherein the first vaccine composition is administered prior to or during the fattening period of said animal to cause a reduction in circulating testosterone levels; and

(b) vaccinating the animal with a second vaccine composition comprising an immunological adjuvant and a GnRH multimer comprising the amino acid sequence depicted in FIGS. 3A-3F (SEQ ID NO:______), or an amino acid sequence with at least about 75% sequence identity thereto, wherein the second vaccine composition is administered at about 2 to about 8 weeks before slaughter of the animal, to substantially reduce the level of one or more androgenic and/or non-androgenic steroids. The adjuvant in the first and/or second vaccine composition may comprise a light mineral oil and dimethyldioctadecylammonium bromide.

These and other embodiments of the present invention will readily occur to those of ordinary skill in the art in view of the disclosure herein.

### BRIEF DESCRIPTION OF THE FIGURES

FIG. 1 depicts the relationship between antibody titers before booster vaccination on Day 35 of the trial when pigs were 63 days of age, and 14 days after booster injection at Day 49 of the trial, when animals were 77 days of age, as described in the examples.

FIGS. 2A and 2B show the nucleotide sequences and amino acid sequences of the GnRH constructs used in the chimeric leukotoxin-GnRH polypeptide gene fusions herein. FIG. 2A depicts a single copy of a GnRH decapeptide. FIG. 2B depicts a molecule with four copies of a GnRH decapeptide when n=1, and eight copies of GnRH when n=2, etc.

FIGS. 3A through 3F show the nucleotide sequence and predicted amino acid sequence of the LKT-GnRH chimeric protein from plasmids pCB122 and pCB130.

FIG. 4 shows body weight as a function of age in pigs treated with GnRH vaccines according to the invention (immunocastrates), as compared to castrated male pigs (barrows) and uncastrated male pigs (boars).

### DETAILED DESCRIPTION

The practice of the present invention will employ, unless otherwise indicated, conventional techniques of molecular biology, microbiology, virology, recombinant DNA technology, and immunology, which are within the skill of the art. Such techniques are explained fully in the literature. See, e.g., Sambrook, Fritsch & Maniatis, Molecular Cloning: A Laboratory Manual; DNA Cloning, Vols. I and II (D. N. Glover ed.); Oligonucleotide Synthesis (M. J. Gait ed.); Nucleic Acid Hybridization (B. D. Hames & S. J. Higgins eds.); B. Perbal, A Practical Guide to Molecular Cloning; the series, Methods In Enzymology (S. Colowick and N. Kaplan eds., Academic Press, Inc.); and Handbook of Experimental Immunology, Vols. I-IV (D. M. Weir and C. C. Blackwell eds., Blackwell Scientific Publications).

All patents, patent applications, and publications mentioned herein, whether supra or infra, are hereby incorporated by reference in their entirety.

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

1. Definitions

In describing the present invention, the following terms will be employed, and are intended to be defined as indicated below.

The term “Gonadotropin releasing hormone” or “GnRH” refers to a decapeptide secreted by the hypothalamus which controls release of both luteinizing hormone (LH) and follicle stimulating hormone (FSH) in vertebrates (Fink, G., British Medical Bulletin (1979) 35:155-160). The amino acid sequence of GnRH is highly conserved among vertebrates, and especially in mammals. In this regard, GnRH derived from most mammals including human, bovine, porcine and ovine GnRH (formerly designated LHRH) has the amino acid sequence pyroGlu-His-Trp-Ser-Tyr-Gly-Leu-Arg-Pro-Gly-NH₂ (SEQ ID NO:1) (Murad et al., Hormones and Hormone Antagonists, in The Pharmacological Basis of Therapeutics, Sixth Edition (1980) and Seeburg et al., Nature (1984) 311:666-668).

As used herein a “GnRH polypeptide” includes a molecule derived from a native GnRH sequence, as well as recombinantly produced or chemically synthesized GnRH polypeptides having amino acid sequences which are substantially homologous to native GnRH and which remain immunogenic, as described below. Thus, the term encompasses derivatives and analogues of GnRH including any single or multiple amino acid additions, substitutions and/or deletions occurring internally or at the amino- or carboxy-termini of the peptide. Accordingly, under the invention, a “GnRH polypeptide” includes molecules having the native sequence as well as analogues of GnRH.

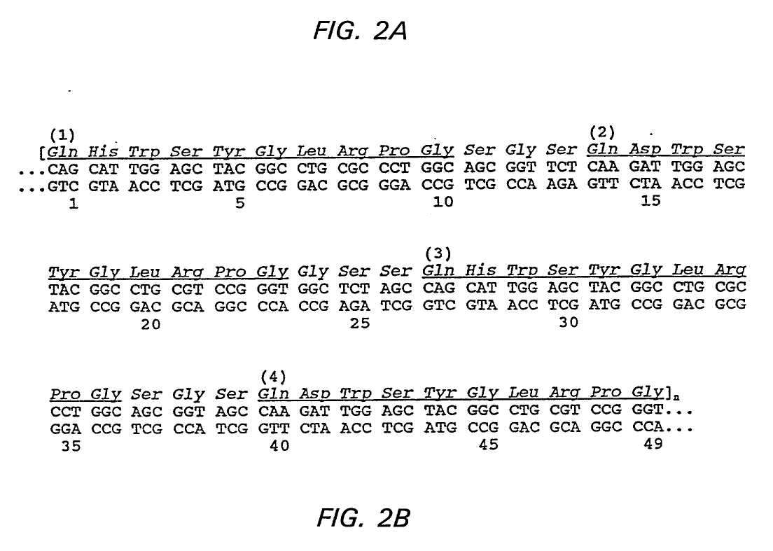

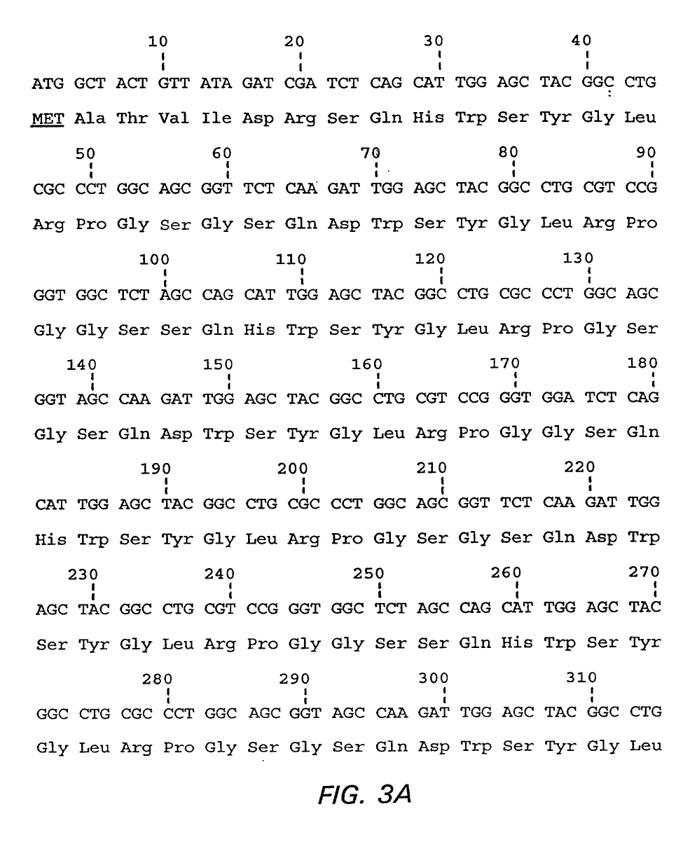

Representative GnRH analogues include an analogue with an N-terminal Gln or Glu residue rather than a pyroGlu residue, an analogue having Asp at amino acid position 2 instead of His (see FIGS. 2A and 2B); a GnRH analogue with an N-terminal addition such as Cys-Gly-GnRH (see, e.g., Prendiville et al., J. Animal Sci. (1995) 73:3030-3037); a carboxyl-containing GnRH analogue (see, e.g., Jago et al., J. Animal Sci. (1997) 75:2609-2619; Brown et al., J. Reproduc. Fertil. (1994) 101:15-21); the GnRH analogue (D-Trp6-Pro9-ethyl amide)GnRH (see, e.g., Tilbrook et al., Hormones and Behavior (1993) 27:5-28) or (D-Trp6)GnRH (see, e.g., Chaffaux et al., Recueil de Medecine Veterinaire (1985) 161:133-145); GnRH analogues with the first, sixth and/or tenth normally occurring amino acids replaced by Cys and/or wherein the N-terminus is acetylated and/or the C-terminus is amidated (see, e.g., U.S. Pat. Nos. 4,608,251 and 4,975,420); the GnRH analogue pyroGlu-His-Trp-Ser-Tyr-X-Leu-Arg-Pro-Gly-Y-Z (SEQ ID NO:______) wherein X is Gly or a D-amino acid, Y is one or more amino acid residues which may be the same or different, preferably 1-3 Gly residues, and Z is Cys or Tyr (see, UK Patent Publication No. GB 2196969); GnRH analogues described in U.S. Pat. No. 5,688,506, including the GnRH analogue Cys-Pro-Pro-Pro-Pro-Ser-Ser-Glu-His-Trp-Ser-Tyr-Gly-Leu-Arg-Pro-Gly (SEQ ID NO:______), pyroGlu-His-Trp-Ser-Tyr-Gly-Leu-Arg-Pro-Gly-Ser-Ser-Pro-Pro-Pro-Pro-Cys (SEQ ID NO:______), pyroGlu-His-Trp-Ser-Tyr-Gly-Leu-Arg-Pro-Gly-Arg-Pro-Pro-Pro-Pro-Cys (SEQ ID NO:______); the GnRH analogue known as Deslorelin, commercially available from Apeptech (Australia), and Ovuplant™; and molecules with other amino acid additions, substitutions and/or deletions which retain the ability to elicit formation of antibodies that cross-react with naturally occurring GnRH.

Thus, the term “GnRH polypeptide” includes a GnRH molecule differing from the reference sequence by having one or more amino acid substitutions, deletions and/or additions and which has at least about 50% amino acid identity to the reference molecule, more preferably about 75-85% identity and most preferably about 90-95% identity or more, to the relevant portion of the native polypeptide sequence in question. The amino acid sequence will have not more than about 1-5 amino acid substitutions, or not more than about 1-3 amino acid substitutions. Particularly preferred substitutions will generally be conservative in nature, i.e., those substitutions that take place within a family of amino acids. In this regard, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cystine, serine threonine, tyrosine. Phenylalanine, tryptophan, and tyrosine are sometimes classified as aromatic amino acids. For example, it is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the activity. Proteins having substantially the same amino acid sequence as the reference molecule, but possessing minor amino acid substitutions that retain the desired activity, are therefore within the definition of a GnRH polypeptide.

A “GnRH polypeptide” also includes peptide fragments of the reference GnRH molecule, so long as the molecule retains the desired activity. Epitopes of GnRH are also captured by the definition.

Particularly contemplated herein are multimers of GnRH including repeating sequences of GnRH polypeptides such as multimers including 2, 4, 8, 16, 32 copies, etc. of one or more GnRH polypeptides, optionally including spacer sequences, such as those described in International Publication Nos. WO 98/06848 and WO 96/24675 and shown in FIG. 2B herein. Such multimers are described more fully below.

For purposes of the present invention, a GnRH polypeptide may be derived from any of the various known GnRH sequences, described above, including without limitation, GnRH polypeptides derived from human, bovine, porcine, ovine, canine, feline, cervine subjects, rodents such as hamsters, guinea pigs, gerbils, ground hogs, gophers, lagomorphs, rabbits, ferrets, squirrels, reptilian and avian subjects.

A “GnRH peptide” is a GnRH polypeptide, as described herein, which includes less than the full-length of the reference GnRH molecule in question and which includes at least one epitope as defined below. Thus, a vaccine composition comprising a GnRH peptide would include a portion of the full-length molecule but not the entire GnRH molecule in question. Particular GnRH peptides for use herein include, for example, GnRH peptides with 5, 6 or 7 amino acids, particularly those peptides which include the amino terminus or the carboxy terminus, such as GnRH peptides including amino acids 1-5, 1-6, 1-7, 2-8, 3-8, 3-10, 4-10 and 5-10 of the native sequence (see, e.g., International Publication No. WO 88/05308).

By “GnRH multimer” is meant a molecule having more than one copy of a selected GnRH polypeptide, GnRH immunogen, GnRH peptide or epitope, or multiple tandem repeats of a selected GnRH polypeptide, GnRH immunogen, GnRH peptide or epitope. The GnRH multimer may correspond to a molecule with repeating units of the general formula (GnRH-X-GnRH)y wherein GnRH is a GnRH polypeptide, X is one or more molecules selected from the group consisting of a peptide linkage, an amino acid spacer group, a carrier molecule and [GnRH]ₙ, where n is an integer greater than or equal to 1, y is an integer greater than or equal to 1, and further wherein “GnRH” may comprise any GnRH polypeptide. Y may therefore define 1-40 or more repeating units, more preferably, 1-30 repeating units and most preferably, 1-20 repeating units. Further, the selected GnRH sequences may all be the same, or may correspond to different derivatives, analogues, variants or epitopes of GnRH, so long as they retain the ability to elicit an immune response. Additionally, if the GnRH units are linked either chemically or recombinantly to a carrier, GnRH molecules may be linked to either the 5′-end, the 3′-end, or may flank the carrier in question. Further, the GnRH multimer may be located at sites internal to the carrier. GnRH multimers are discussed in further detail below.

The term “GnRH immunogen” refers to GnRH polypeptides, as described above, that elicit an immunological response without an associated immunological carrier, adjuvant or immunostimulant, as well as GnRH polypeptides capable of being rendered immunogenic, or more immunogenic, by way of association with a carrier molecule, adjuvant or immunostimulant, or by mutation of a native sequence, and/or by incorporation into a molecule containing multiple repeating units of at least one epitope of a GnRH molecule. The term may be used to refer to an individual macromolecule or to a homogeneous or heterogeneous population of antigenic macromolecules derived from GnRH.

Generally, a GnRH immunogen will elicit formation of antibodies that cross-react with the naturally occurring, endogenous GnRH of the vertebrate species to which such an immunogen is delivered. The term “GnRH immunogen” also refers to nucleic acid molecules, such as DNA and RNA molecules encoding GnRH polypeptides which are capable of expression in vivo, when administered using nucleic acid delivery techniques described further below.

“Homology” refers to the percent identity between two polynucleotide or two polypeptide moieties. Two DNA, or two polypeptide sequences are “substantially homologous” to each other when the sequences exhibit at least about 75%-85%, preferably at least about 90%, and most preferably at least about 95%-98% sequence identity over a defined length of the molecules. As used herein, substantially homologous also refers to sequences showing complete identity to the specified DNA or polypeptide sequence.

Percent “identity” between two amino acid or polynucleotide sequences can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman (1981) Advances in Appl. Math. 2:482-489 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

An “epitope” refers to any portion or region of a molecule with the ability or potential to elicit, and combine with, a GnRH-specific antibody. For purposes of the present invention, a polypeptide epitope will usually include at least about 3 amino acids, preferably at least about 5 amino acids of the reference molecule. There is no critical upper limit to the length of the fragment, which could comprise nearly the full-length of a protein sequence, or even a fusion protein comprising two or more epitopes of a protein in question.

Because GnRH is a very small molecule, the identification of epitopes thereof which are able to elicit an antibody response is readily accomplished using techniques well known in the art. For example, epitopes in polypeptide molecules can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra.

Computer programs that formulate hydropathy scales from the amino acid sequence of the protein, utilizing the hydrophobic and hydrophilic properties of each of the 20 amino acids, as described in, e.g., Kyte et al., J. Mol. Biol. (1982) 157:105-132; and Hopp and Woods, Proc. Natl. Acad. Sci. USA (1981) 78:3824-3828, can also be used to determine antigenic portions of a given molecule. For example, the technique of Hopp and Woods assigns each amino acid a numerical hydrophilicity value and then repetitively averages these values along the peptide chain. The points of highest local average hydrophilicities are indicative of antigenic portions of the molecule.

By “immunological carrier” is meant any molecule which, when associated with a GnRH immunogen of interest, imparts immunogenicity to that molecule, or enhances the immunogenicity of the molecule. Examples of suitable carriers include large, slowly metabolized macromolecules such as: proteins; polysaccharides, such as sepharose, agarose, cellulose, cellulose beads and the like; polymeric amino acids such as polyglutamic acid, polylysine, and the like; amino acid copolymers; inactive virus particles; bacterial toxins such as toxoid from diphtheria, tetanus, cholera, leukotoxin molecules, and the like. Carriers are described in further detail below.

A GnRH immunogen is “linked” to a specified carrier molecule when the immunogen is chemically coupled to, or associated with the carrier, or when the immunogen is expressed from a chimeric DNA molecule which encodes the immunogen and the carrier of interest.

An “immunoconjugate” is a GnRH immunogen such as a GnRH peptide or multimer which is linked to a carrier molecule, as defined above.

The term “leukotoxin polypeptide” or “LKT polypeptide” intends a polypeptide which is derived from a protein belonging to the family of molecules characterized by the carboxy-terminus consensus amino acid sequence Gly-Gly-X-Gly-X-Asp (Highlander et al. (1989) DNA 8:15-28), wherein X is Lys, Asp, Val or Asn. Such proteins include, among others, leukotoxins derived from P. haemolytica and Actinobacillus pleuropneumoniae, as well as E. coli alpha hemolysin (Strathdee et al. (1987) Infect. Immun. 55:3233-3236; Lo (1990) Can. J. Vet. Res. 54:S33-S35; Welch (1991) Mol. Microbiol. 5:521-528). This family of toxins is known as the “RTX” family of toxins (Lo (1990) Can. J. Vet. Res. 54:S33-S35). In addition, the term “leukotoxin polypeptide” refers to a leukotoxin polypeptide which is chemically synthesized, isolated from an organism expressing the same, or recombinantly produced. Furthermore, the term intends an immunogenic protein having an amino acid sequence substantially homologous to a contiguous amino acid sequence found in the particular native leukotoxin molecule. Thus, the term includes both full-length and partial sequences, as well as analogues. Although native full-length leukotoxins display cytotoxic activity, the term “leukotoxin” also intends molecules which remain immunogenic yet lack the cytotoxic character of native leukotoxins.

The nucleotide sequences and corresponding amino acid sequences for several leukotoxins are known. See, e.g., U.S. Pat. Nos. 4,957,739 and 5,055,400; Lo et al. (1985) Infect. Immun. 50:667-67; Lo et al. (1987) Infect. Immun. 55:1987-1996; Strathdee et al. (1987) Infect. Immun. 55:3233-3236; Highlander et al. (1989) DNA 8:15-28; and Welch (1991) Mol. Microbiol. 5:521-528. In preferred embodiments of the invention, leukotoxin chimeras are provided having a selected leukotoxin polypeptide sequence that imparts enhanced immunogenicity to one or more GnRH multimers fused thereto.

Particular examples of immunogenic leukotoxin polypeptides for use in the present invention are truncated leukotoxin molecules described in U.S. Pat. Nos. 5,476,657 and 5,837,268, incorporated herein by reference in their entireties. These truncated molecules include LKT 352, LKT 111 and LKT 114. LKT 352 is derived from the lktA gene present in plasmid pAA352 (ATCC Accession No. 68283). The nucleotide sequence and corresponding amino acid sequence of this gene are described in U.S. Pat. No. 5,476,657. The gene encodes a truncated leukotoxin, having 914 amino acids and an estimated molecular weight of around 99 kDa. LKT 111 is a leukotoxin polypeptide derived from the lktA gene present in plasmid pCB111 (ATCC Accession No. 69748). The nucleotide sequence of this gene and the corresponding amino acid sequence are disclosed in U.S. Pat. No. 5,837,268. The gene encodes a shortened version of leukotoxin which was developed from the recombinant leukotoxin gene present in plasmid pAA352 (ATCC Accession No. 68283) by removal of an internal DNA fragment of approximately 1300 bp in length. The LKT 111 polypeptide has an estimated molecular weight of 52 kDa (as compared to the 99 kDa LKT 352 polypeptide), but retains portions of the LKT 352 N-terminus containing T-cell epitopes which are necessary for sufficient T-cell immunogenicity, and portions of the LKT 352 C-terminus containing convenient restriction sites for use in producing fusion proteins for use in the present invention. LKT 114 is derived from the gene present in plasmid pAA114 (described in U.S. Pat. No. 5,837,268). LKT 114 differs from LKT 111 by virtue of an additional amino acid deletion from the internal portion of the molecule.

By “immunological adjuvants” is meant an agent which acts in a nonspecific manner to increase an immune response to a particular antigen, thus reducing the quantity of antigen necessary in any given vaccine, and/or the frequency of injection necessary in order to generate an adequate immune response to the antigen of interest. See, e.g., A. C. Allison J. Reticuloendothel. Soc. (1979) 26:619-630.

“Native” proteins, polypeptides or peptides are proteins, polypeptides or peptides isolated from the source in which the proteins naturally occur. “Recombinant” polypeptides refer to polypeptides produced by recombinant DNA techniques; i.e., produced from cells transformed by an exogenous DNA construct encoding the desired polypeptide. “Synthetic” polypeptides are those prepared by chemical synthesis.

By “polynucleotide” is meant a sequence of nucleotides including, but is not limited to, RNA such as mRNA, cDNA, genomic DNA sequences and even synthetic DNA sequences. The term also captures sequences that include any of the known base analogues of DNA and RNA.

The term “derived from,” as it is used herein, denotes an actual or theoretical source or origin of the subject molecule or immunogen. For example, an immunogen that is “derived from” a particular GnRH molecule will bear close sequence similarity with a relevant portion of the reference molecule. Thus, an immunogen that is “derived from” a particular GnRH molecule may include all of the wild-type GnRH sequence, or may be altered by insertion, deletion or substitution of amino acid residues, so long as the derived sequence provides for an immunogen that corresponds to the targeted GnRH molecule. Immunogens derived from a denoted molecule will contain at least one epitope specific to the denoted molecule.

By “food-producing animal” is meant an animal intended for consumption by humans or domestic pets such as cats and dogs. Such animals include, without limitation, mammals such as ovine, bovine, porcine, and cervine subjects, including sheep, cattle, pigs and deer.

By “enhancing the organoleptic qualities of meat” is meant improving the smell, taste and/or tenderness of meat from an animal treated under the invention as compared to meat from a typical uncastrated member of the same species that has not been so treated. Meat from uncastrated males generally suffers from several drawbacks. In this regard, meat derived from uncastrated male pigs and sheep often has an unpleasant taste and smell. For example, “boar taint” refers to a urine-like odor found in cooked meat of uncastrated pigs. Boar taint is produced by steroids stored in tissues in male piglets with normally functioning testicles. See e.g. Brooks et al., J. Anim. Sci. (1986) 62:1279. The presence of androstenone in boar carcasses is one measure of boar taint and is considered a measure of gonadal steroid production. Additionally, skatole may contribute to boar taint. See, e.g., Mortensen and Sorensen, Proc. 30th European Meeting of Meat Research Workers, Ghent, Belgium (1986), pp. 394-396 for a method for assaying for skatole in fat. Similarly, uncastrated male cattle often produce leaner but tougher meat, by virtue of the increased muscle mass. Thus, meat with improved organoleptic properties is meat with a more desirable smell, tenderness and/or taste.

By a “reduction in circulating testosterone” is meant a statistically significant reduction in serum testosterone levels as measured using a standard assay, such as an RIA as described herein, as compared with the serum testosterone levels expected in a typical uncastrated, untreated male, of the same age and species.

“Androgenic” steroids include androstenone, androstenedione, androstenediol and/or testosterone. Androgenic steroids can be measured using well known techniques. For example, testosterone and the other androgenic steroids can be measured using ELISAs and RIAs well known in the art. Particularly convenient measures may be made using commercially available test kits, e.g., the Coat-A-Count Total Testosterone Kit™ (Diagnostic Products Corporation, Los Angeles, Calif.). This kit is a solid-phase RIA designed for the quantitative measurement of testosterone in serum, based on testosterone-specific antibody immobilized to the wall of a polypropylene tube. See, also Schanbacher and D'Occhio, J. Andrology (1982) 3:45-51, for a description of a direct RIA for determining testosterone levels. ELISAs for determining androstenone levels are described in, e.g., Abouzied et al., J. Agri. Food Chem. (1990) 38:331-335. See, also Meloen et al., Vaccine (1994) 12(8):741-746; and Booth et al., Anim. Prod. (1986) 42:145-152 describing ELISAs done on androstenone extracted from fat. RIAs for determining androstenone levels are also known. See, e.g., Andersen, O., Acta. Vet. Scand. (1979) 20:343-350.

“Non-androgenic” steroids include the 16-androstene derivatives, including 5αandrostenone (5αandrost-16-en-3-one). Non-androgenic steroids can be measured using techniques well known in the art, such as by ELISAs and RIAs. See, e.g., Claus et al., Archiv fuer Lebensmittelhygiene (1988) 39:87-90.

By a “substantially reduced” level of one or more androgenic and/or non-androgenic steroids is meant that the level of at least one androgenic or non-adrogenic steroid is at least about 50% less than expected in a typical uncastrated, untreated male, of the same age and species, preferably at least about 75% less, and more preferably at least about 80% to 90% or less.

The term “fattening period” intends the period from weaning up to slaughter and thus includes the pre-, peri- and post-pubertal periods. A typical fattening period will vary from species to species and even within a species, depending on the preference of the food-producer and the country where the animals are raised. Thus, the fattening period is largely a matter of choice and one of skill in the art can readily determine the appropriate fattening period for a given animal.

2. General Methods

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of compositions and methods similar or equivalent to those described herein can be used in the practice of the present invention, the preferred materials and methods are described herein.

Central to the instant invention is the discovery of a method for improving meat quality by modulating gonadal steroid secretion. The method includes one or more primary immunizations before or during the fattening period of the animal with a GnRH formulation designed to cause a measurable reduction of circulating testosterone levels, but generally does not result in complete immunocastration. The primary vaccination is followed with a boost with the same or different GnRH composition shortly before slaughter, to substantially reduce the level of one or more androgenic and/or non-androgenic steroids.

Although GnRH is generally recognized as “self” and hence nonimmunogenic, the compositions described herein surprisingly provide a means for producing an adequate immunological response in a subject immunized therewith.

The timing of the vaccinations depends on the animal in question which is generally a sheep, cow or pig, as well as the preference of the food-producer. However, the first vaccination will be given prior or during the fattening period of the animal. For example, in pigs and sheep, the primary immunization will generally be given at a time between the birth of the animal and about 15 weeks of age, preferably at a time between the birth of the animal and about 10 weeks of age. In cows, the primary immunization will generally be given at a time between birth and about 48 weeks of age.

One or more booster treatments are given before slaughter. The timing of the booster will also depend on the animal in question. For example, in pigs and sheep, the booster will generally be at about 1 to about 12 weeks prior to slaughter, preferably about 2 to about 8 weeks prior to slaughter and most preferably about 4 to about 6 weeks prior to slaughter, and even 2 to about 3 weeks prior to slaughter. In cows, it may be preferable to administer the second vaccine composition several months prior to slaughter.

In certain embodiments, the subsequent immunization(s) is given after GnRH antibodies, raised against the primary immunization, have declined, i.e., to a level at least about 50% below the maximum antibody levels detected, preferably decreased at least about 75% below the maximum levels detected.

The vaccine compositions of the present invention employ GnRH polypeptides, as defined above, optionally linked to carrier molecules in order to enhance immunogenicity thereof.

GnRH Immunoconjugates

As explained above, GnRH is an endogenous molecule and, as such, it may be desirable to further increase the immunogenicity of the GnRH polypeptides (or multimers described below) by linking them to carriers to form GnRH immunoconjugates. This is especially necessary if the GnRH immunogen will be administered to the same species from which it is derived.

Suitable carriers are generally polypeptides which include antigenic regions of a protein derived from an infectious material such as a viral surface protein, or a carrier peptide sequence. These carriers serve to non-specifically stimulate T-helper cell activity and to help direct an immunogen of interest to antigen presenting cells (APCs) for processing and presentation at the cell surface in association with molecules of the major histocompatibility complex (MHC).

Several carrier systems have been developed for this purpose. For example, small peptide haptens are often coupled to protein carriers such as keyhole limpet hemocyanin (Bittle et al. (1982) Nature 298:30-33), bacterial toxins such as tetanus toxoid (Muller et al. (1982) Proc. Natl. Acad. Sci. U.S.A. 79:569-573), ovalbumin, leukotoxin polypeptides, and sperm whale myoglobin, to produce an immune response. These coupling reactions typically result in the incorporation of several moles of peptide hapten per mole of carrier protein.

Other suitable carriers for use with the present invention include VP6 polypeptides of rotaviruses, or functional fragments thereof, as disclosed in U.S. Pat. No. 5,071,651. Also useful is a fusion product of a viral protein and one or more epitopes from GnRH, which fusion products are made by the methods disclosed in U.S. Pat. No. 4,722,840. Still other suitable carriers include cells, such as lymphocytes, since presentation in this form mimics the natural mode of presentation in the subject, which gives rise to the immunized state. Alternatively, the GnRH immunogens may be coupled to erythrocytes, preferably the subject's own erythrocytes. Methods of coupling peptides to proteins or cells are known to those of skill in the art.

Delivery systems useful in the practice of the present invention may also utilize particulate carriers. For example, pre-formed particles have been used as platforms onto which immunogens can be coupled and incorporated. Systems based on proteosomes (Lowell et al. (1988) Science 240:800-802) and immune stimulatory complexes (Morein et al. (1984) Nature 308:457-460) are also known in the art.

Carrier systems using recombinantly produced chimeric proteins that self-assemble into particles may also be used with the present invention. For example, the yeast retrotransposon, Ty, encodes a series of proteins that assemble into virus like particles (Ty-VLPs; Kingsman et al. (1988) Vaccines 6:304-306). Thus, a gene, or fragment thereof, encoding the GnRH immunogen of interest may be inserted into the TyA gene and expressed in yeast as a fusion protein. The fusion protein retains the capacity to self assemble into particles of uniform size. Other useful virus-like carrier systems are based on HBsAg, (Valenzuela et al. (1985) Bio/Technol. 3:323-326; U.S. Pat. No. 4,722,840; Delpeyroux et al. (1986) Science 233:472-475), Hepatitis B core antigen (Clarke et al. (1988) Vaccines 88 (Ed. H. Ginsberg, et al.) pp. 127-131), Poliovirus (Burke et al. (1988) Nature 332:81-82), and Tobacco Mosaic Virus (Haynes et al. (1986) Bio/Technol. 4:637-641).

Especially preferred carriers include serum albumins, keyhole limpet hemocyanin, ovalbumin, sperm whale myoglobin, leukotoxin molecules as described above, and other proteins well known to those skilled in the art. For example, chimeric systems using a leukotoxin polypeptide, as defined above, such as a Pasteurella haemolytica leukotoxin (LKT) polypeptide fused to the antigen of interest, can also be used herein. In this regard, the nucleotide sequences and corresponding amino acid sequences for several leukotoxin carriers are known. See, e.g., U.S. Pat. Nos. 5,422,110, 5,708,155, 5,723,129 and International Publication Nos. WO 98/06848 and WO 96/24675. Particular examples of immunogenic leukotoxin polypeptides for use herein include LKT 342, LKT 352, LKT 111, LKT 326 and LKT 101 which are described in the patents and publications cited above. Particularly preferred are LKT 111 and LKT 114. The gene encoding LKT 111 was developed from the recombinant leukotoxin gene present in plasmid pAA352 (ATCC Accession No. 68283) by removal of an internal DNA fragment of approximately 1300 bp in length. The LKT 111 polypeptide has an estimated molecular weight of 52 kDa (as compared to the 99 kDa LKT 352 polypeptide), but retains portions of the LKT 352 N-terminus containing T-cell epitopes which are necessary for sufficient T-cell immunogenicity, and portions of the LKT 352 C-terminus containing convenient restriction sites for use in producing the fusion proteins of the present invention. LKT 114 differs from LKT 111 by virtue of an additional amino acid deletion from the internal portion of the molecule. See, e.g., U.S. Pat. No. 5,837,268 and International Publication Nos. WO 98/06848 and WO 96/24675 for descriptions of these molecules.

Protein carriers may be used in their native form or their functional group content may be modified by, for example, succinylation of lysine residues or reaction with Cys-thiolactone. A sulfhydryl group may also be incorporated into the carrier (or antigen) by, for example, reaction of amino functions with 2-iminothiolane or the N-hydroxysuccinimide ester of 3-(4-dithiopyridyl propionate. Suitable carriers may also be modified to incorporate spacer arms (such as hexamethylene diamine or other bifunctional molecules of similar size) for attachment of peptide immunogens.

Carriers can be physically conjugated to the GnRH immunogen of interest, using standard coupling reactions. Alternatively, chimeric molecules can be prepared recombinantly for use in the present invention, such as by fusing a gene encoding a suitable polypeptide carrier to one or more copies of a gene, or fragment thereof, encoding for a selected GnRH immunogen. The GnRH portion can be fused either 5′ or 3′ to the carrier portion of the molecule, or the GnRH portion may be located at sites internal to the carrier molecule.

The GnRH immunogens can also be administered via a carrier virus which expresses the same. Carrier viruses which will find use herein include, but are not limited to, the vaccinia and other pox viruses, adenovirus, and herpes virus. By way of example, vaccinia virus recombinants expressing the proteins can be constructed as follows. The DNA encoding a particular protein is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the desired immunogen into the viral genome. The resulting TK-recombinant can be selected by culturing the cells in the presence of 5-bromodeoxyuridine and picking viral plaques resistant thereto.

GnRH Multimers

Immunogenicity of the GnRH immunogens may also be significantly increased by producing immunogenic forms of the molecules that comprise multiple copies of selected epitopes. In this way, endogenous GnRH may be rendered an effective autoantigen.

Accordingly, in one aspect of the invention, vaccine compositions containing GnRH immunogen multimers are provided in either nucleic acid or peptide form. The GnRH multimer will have more than one copy of selected GnRH immunogens, peptides or epitopes, as described above, or multiple tandem repeats of a selected GnRH immunogen, peptide or epitope. Thus, the GnRH multimers may comprise either multiple or tandem repeats of selected GnRH sequences, multiple or tandem repeats of selected GnRH epitopes, or any conceivable combination thereof. GnRH epitopes may be identified using techniques as described in detail above.

For example, the GnRH multimer may correspond to a molecule with repeating units of the general formula (GnRH-X-GnRH)y wherein GnRH is a GnRH immunogen, X is selected from the group consisting of a peptide linkage, an amino acid spacer group, a carrier molecule and [GnRH]ₙ, where n is an integer greater than or equal to 1, y is an integer greater than or equal to 1, and further wherein “GnRH” may comprise any GnRH immunogen. Thus, the GnRH multimer may contain from 2-64 or more GnRH immunogens, more preferably 2-32 or 2-16 GnRH immunogens.

Further, the selected GnRH immunogen sequences may all be the same, or may correspond to different derivatives, analogues, variants or epitopes of GnRH so long as they retain the ability to elicit an immune response. Additionally, if the GnRH immunogens are linked either chemically or recombinantly to a carrier, GnRH immunogens may be linked to either the 5′-end, the 3′-end, or may flank the carrier in question. Further, the GnRH multimer may be located at sites internal to the carrier. One particular carrier for use with the present GnRH multimers is a leukotoxin polypeptide as described above.

As explained above, spacer sequences may be present between the GnRH moieties. For example, Ser-Gly-Ser trimers and Gly-Ser dimers are present in the GnRH multimers exemplified herein which provide spacers between repeating sequences of the GnRH immunogens. See, e.g., FIG. 2B. The strategic placement of various spacer sequences between selected GnRH immunogens can be used to confer increased immunogenicity on the subject constructs. Accordingly, under the invention, a selected spacer sequence may encode a wide variety of moieties such as a single amino acid linker or a sequence of two to several amino acids. Selected spacer groups may preferably provide enzyme cleavage sites so that the expressed multimer can be processed by proteolytic enzymes in vivo (by APCs, or the like) to yield a number of peptides, each of which contain at least one T-cell epitope derived from the carrier portion, and which are preferably fused to a substantially complete GnRH polypeptide sequence.

The spacer groups may be constructed so that the junction region between selected GnRH moieties comprises a clearly foreign sequence to the immunized subject, thereby conferring enhanced immunogenicity upon the associated GnRH immunogens. Additionally, spacer sequences may be constructed so as to provide T-cell antigenicity, such as those sequences which encode amphipathic and/or α-helical peptide sequences which are generally recognized in the art as providing immunogenic helper T-cell epitopes. The choice of particular T-cell epitopes to be provided by such spacer sequences may vary depending on the particular vertebrate species to be vaccinated. Although particular GnRH portions are exemplified which include spacer sequences, it is also an object of the invention to provide one or more GnRH multimers comprising directly adjacent GnRH sequences (without intervening spacer sequences).

The GnRH multimeric sequence thus produced renders a highly immunogenic GnRH antigen for use in the compositions of the invention.

The GnRH polypeptides, immunoconjugates and multimers can be produced using the methods described below, and used for nucleic acid immunization, gene therapy, protein-based immunization methods, and the like.

Nucleic Acid-Based Immunization Methods

Generally, nucleic acid-based vaccines for use with the present invention will include relevant regions encoding a GnRH immunogen, with suitable control sequences and, optionally, ancillary therapeutic nucleotide sequences. The nucleic acid molecules are prepared in the form of vectors which include the necessary elements to direct transcription and translation in a recipient cell.

In order to augment an immune response in an immunized subject, the nucleic acid molecules can be administered in conjunction with ancillary substances, such as pharmacological agents, adjuvants, or in conjunction with delivery of vectors encoding biological response modifiers such as cytokines and the like. Other ancillary substances include, but are not limited to, substances to increase weight gain, muscle mass or muscle strength, such as growth hormones, growth promoting agents, beta antagonists, partitioning agents and antibiotics.

Nucleotide sequences selected for use in the present invention can be derived from known sources, for example, by isolating the same from cells or tissue containing a desired gene or nucleotide sequence using standard techniques, or by using recombinant or synthetic techniques.

Once coding sequences for the GnRH immunogens have been prepared or isolated, such sequences can be cloned into any suitable vector or replicon. Numerous cloning vectors are known to those of skill in the art, and the selection of an appropriate cloning vector is a matter of choice. Ligations to other sequences, e.g., ancillary molecules or carrier molecules, are performed using standard procedures, known in the art. One or more GnRH immunogen portions of the chimera can be fused 5′ and/or 3′ to a desired ancillary sequence or carrier molecule. Alternatively, one or more GnRH immunogen portions may be located at sites internal to the carrier molecule, or such portions can be positioned at both terminal and internal locations in the chimera.

Alternatively, DNA sequences encoding the GnRH immunogens of interest, optionally linked to carrier molecules, can be prepared synthetically rather than cloned. The DNA sequences can be designed with appropriate codons for the particular sequence. The complete sequence of the immunogen is then assembled from overlapping oligonucleotides prepared by standard methods and assembled into a complete coding sequence. See, e.g., Edge (1981) Nature 292:756; Nambair et al. (1984) Science 223:1299; and Jay et al. (1984) J. Biol. Chem. 259:6311.

The coding sequence is then placed under the control of suitable control elements for expression in suitable host tissue in vivo. The choice of control elements will depend on the subject being treated and the type of preparation used. Thus, if the subject's endogenous transcription and translation machinery will be used to express the immunogens, control elements compatible with the particular subject will be utilized. In this regard, several promoters for use in mammalian systems are known in the art. For example, typical promoters for mammalian cell expression include the SV40 early promoter, a CMV promoter such as the CMV immediate early promoter, the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression.

Typically, transcription termination and polyadenylation sequences will also be present, located 3′ to the translation stop codon. Preferably, a sequence for optimization of initiation of translation, located 5′ to the coding sequence, is also present. Examples of transcription terminator/polyadenylation signals include those derived from SV40, as described in Sambrook et al., supra, as well as a bovine growth hormone terminator sequence. Introns, containing splice donor and acceptor sites, may also be designed into the constructs for use with the present invention.

Enhancer elements may also be used herein to increase expression levels of the constructs. Examples include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777) and elements derived from human CMV (Boshart et al. (1985) Cell 41:521), such as elements included in the CMV intron A sequence.

Once prepared, the nucleic acid vaccine compositions can be delivered to the subject using known methods. In this regard, various techniques for immunization with antigen-encoding DNAs have been described. See, e.g., U.S. Pat. No. 5,589,466 to Felgner et al.; Tang et al. (1992) Nature 358:152; Davis et al. (1993) Hum. Molec. Genet. 2:1847; Ulmer et al. (1993) Science 258:1745; Wang et al. (1993) Proc. Natl. Acad. Sci. USA 90:4156; Eisenbraun et al. (1993) DNA Cell Biol. 12:791; Fynan et al. (1993) Proc. Natl. Acad. Sci. USA 90:12476; Fuller et al. (1994) AIDS Res. Human Retrovir. 10:1433; and Raz et al. (1994) Proc. Natl. Acad. Sci. USA 91:9519. General methods for delivering nucleic acid molecules to cells in vitro, for the subsequent reintroduction into the host, can also be used, such as liposome-mediated gene transfer. See, e.g., Hazinski et al. (1991) Am. J. Respir. Cell Mol. Biol. 4:206-209; Brigham et al. (1989) Am. J. Med. Sci. 298:278-281; Canonico et al. (1991) Clin. Res. 39:219A; and Nabel et al. (1990) Science 249:1285-1288. Thus, the nucleic acid vaccine compositions can be delivered in either liquid or particulate form using a variety of known techniques. Typical vaccine compositions are described more fully below.

Protein-Based Delivery Methods

Protein-based compositions can also be produced using a variety of methods known to those skilled in the art. In particular, GnRH polypeptides can be isolated directly from native sources, using standard purification techniques. Alternatively, the polypeptides can be recombinantly produced using nucleic acid expression systems, well known in the art and described in, e.g., Sambrook et al., supra. GnRH polypeptides can also be synthesized using chemical polymer syntheses such as solid phase peptide synthesis. Such methods are known to those skilled in the art. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques.

GnRH polypeptides for use in the compositions described herein may also be produced by cloning the coding sequences therefor into any suitable expression vector or replicon. Numerous cloning vectors are known to those of skill in the art, and the selection of an appropriate cloning vector is a matter of choice. Examples of recombinant DNA vectors for cloning, and host cells which they can transform, include the bacteriophage lambda (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

For example, the coding sequences for porcine, bovine and ovine GnRH have been determined (Murad et al. (1980) Hormones and Hormone Antagonists, in The Pharmacological Basis of Therapeutics, Sixth Edition), and the cDNA for human GnRH has been cloned so that its sequence has been well established (Seeburg et al. (1984) Nature 311:666-668). Additional GnRH polypeptides of known sequences have been disclosed, such as the GnRH molecule occurring in salmon and chickens (International Publication No. WO 86/07383, published Dec. 18, 1986). Particular GnRH coding sequences for use with the present invention are shown in FIGS. 2A and 2B herein. The GnRH coding sequence is highly conserved in vertebrates, particularly in mammals, and porcine, bovine, ovine and human GnRH sequences are identical to one another.

Portions of these sequences encoding desired GnRH polypeptides, and optionally, a sequence encoding a carrier protein, can be cloned, isolated and ligated together using recombinant techniques generally known in the art. See, e.g., Sambrook et al., supra.

The gene can be placed under the control of a promoter, ribosome binding site (for bacterial expression) and, optionally, an operator, so that the DNA sequence of interest is transcribed into RNA by a suitable transformant. The coding sequence may or may not contain a signal peptide or leader sequence. The polypeptides can be expressed using, for example, the E. coli tac promoter or the protein A gene (spa) promoter and signal sequence. Leader sequences can be removed by the bacterial host in post-translational processing. See, e.g., U.S. Pat. Nos. 4,431,739; 4,425,437; 4,338,397. Ancillary sequences, such as those described above, may also be present.

In addition to control sequences, it may be desirable to add regulatory sequences which allow for regulation of the expression of the polypeptide sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the particular GnRH polypeptide may be desirable to achieve this end. For example, in some cases it may be necessary to modify the sequence so that it can be attached to the control sequences in the appropriate orientation; i.e., to maintain the reading frame. The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

In some cases, it may be desirable to add sequences which cause the secretion of the polypeptide from the host organism, with subsequent cleavage of the secretory signal. It may also be desirable to produce mutants or analogues of the polypeptide. Mutants or analogues may be prepared by the deletion of a portion of the sequence encoding the reference polypeptide, or if present, a portion of the sequence encoding the desired carrier molecule, by insertion of a sequence, and/or by substitution of one or more nucleotides within the sequence. Techniques for modifying nucleotide sequences, such as site-directed mutagenesis, and the like, are well known to those skilled in the art. See, e.g., Sambrook et al., supra; DNA Cloning, Vols. I and II, supra; Nucleic Acid Hybridization, supra; Kunkel, T. A. Proc. Natl. Acad. Sci. USA (1985) 82:448; Geisselsoder et al. BioTechniques (1987) 5:786; Zoller and Smith, Methods Enzymol. (1983) 100:468; Dalbie-McFarland et al. Proc. Natl. Acad. Sci USA (1982) 79:6409.

The GnRH polypeptides can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the GnRH polypeptides are produced by growing host cells transformed by an expression vector described above under conditions whereby the polypeptide is expressed. The expressed polypeptide is then isolated from the host cells and purified. If the expression system secretes the polypeptide into growth media, the product can be purified directly from the media. If it is not secreted, it can be isolated from cell lysates. The selection of the appropriate growth conditions and recovery methods are within the skill of the art.

Once obtained, the GnRH polypeptides, with or without associated carrier, may be formulated into compositions, such as vaccine compositions as described further below, in order to elicit antibody production.

Antibody Production

The subject GnRH immunogens can be used to generate antibodies for use in passive immunization methods. Typically, peptides useful for producing antibodies will usually be at least about 3-5 amino acids in length, preferably 7-10 amino acids in length.

Antibodies against the subject immunogens include polyclonal and monoclonal antibody preparations, monospecific antisera, as well as preparations including hybrid antibodies, altered antibodies, F(ab′)₂ fragments, F(ab) fragments, Fᵥ fragments, single domain antibodies, chimeric antibodies, humanized antibodies, and functional fragments thereof, which retain specificity for the target molecule in question. For example, an antibody can include variable regions, or fragments of variable regions, which retain specificity for the molecule in question. The remainder of the antibody can be derived from the species in which the antibody will be used. Thus, if the antibody is to be used in a human, the antibody can be “humanized” in order to reduce immunogenicity yet retain activity. For a description of chimeric antibodies, see, e.g., Winter, G. and Milstein, C. (1991) Nature 349:293-299; Jones, P. T. et al. (1986) Nature 321:522-525; Riechmann, L. et al. (1988) 332:323-327; and Carter, P. et al. (1992) Proc. Natl. Acad. Sci. USA 89:4285-4289. Such chimeric antibodies may contain not only combining sites for the target molecule, but also binding sites for other proteins. In this way, bifunctional reagents can be generated with targeted specificity to both external and internal antigens.

If polyclonal antibodies are desired, a selected mammal, (e.g., mouse, rabbit, goat, horse, etc.) is immunized with the desired antigen, or its fragment, or a mutated antigen, as described above. Prior to immunization, it may be desirable to further increase the immunogenicity of a particular immunogen. This can be accomplished in any one of several ways known to those of skill in the art.

For example, immunization for the production of antibodies is generally performed by mixing or emulsifying the protein in a suitable excipient, such as saline, preferably in an adjuvant such as Freund's complete adjuvant, or any of the adjuvants described below, and injecting the mixture or emulsion parenterally (generally subcutaneously or intramuscularly). The animal is generally boosted 2-6 weeks later with one or more injections of the protein in saline, preferably using Freund's incomplete adjuvant, or the like. Antibodies may also be generated by in vitro immunization, using methods known in the art. Polyclonal antisera is then obtained from the immunized animal and treated according to known procedures. See, e.g., Jurgens et al. (1985) J. Chrom. 348:363-370. If serum containing polyclonal antibodies is used, the polyclonal antibodies can be purified by immunoaffinity chromatography, using known procedures.

Monoclonal antibodies are generally prepared using the method of Kohler and Milstein, Nature (1975) 256:495-96, or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of non-specifically adherent cells) by applying a cell suspension to a plate or well coated with the protein antigen. B-cells, expressing membrane-bound immunoglobulin specific for the antigen, will bind to the plate, and are not rinsed away with the rest of the suspension. Resulting B-cells, or all dissociated spleen cells, are then induced to fuse with myeloma cells to form hybridomas, and are cultured in a selective medium (e.g., hypo-xanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (as ascites in mice). See, e.g., M. Schreier et al., Hybridoma Techniques (1980); Hammerling et al., Monoclonal Antibodies and T-cell Hybridomas (1981); Kennett et al., Monoclonal Antibodies (1980); see also U.S. Pat. Nos. 4,341,761; 4,399,121; 4,427,783; 4,444,887; 4,452,570; 4,466,917; 4,472,500, 4,491,632; and 4,493,890. Panels of monoclonal antibodies produced against the GnRH immunogen of interest, or fragment thereof, can be screened for various properties; i.e., for isotype, epitope, affinity, etc.

Functional fragments of the antibodies can also be made against the GnRH immunogen of interest and can be produced by cleaving a constant region, not responsible for antigen binding, from the antibody molecule, using e.g., pepsin, to produce F(ab′)₂ fragments. These fragments will contain two antigen binding sites, but lack a portion of the constant region from each of the heavy chains. Similarly, if desired, Fab fragments, comprising a single antigen binding site, can be produced, e.g., by digestion of polyclonal or monoclonal antibodies with papain. Functional fragments, including only the variable regions of the heavy and light chains, can also be produced, using standard techniques. These fragments are known as Fᵥ.

Chimeric or humanized antibodies can also be produced using the subject immunogens. These antibodies can be designed to minimize unwanted immunological reactions attributable to heterologous constant and species-specific framework variable regions typically present in monoclonal and polyclonal antibodies. For example, if the antibodies are to be used in human subjects, chimeric antibodies can be created by replacing non-human constant regions, in either the heavy and light chains, or both, with human constant regions, using techniques generally known in the art. See, e.g., Winter, G. and Milstein, C. (1991) Nature 349:293-299; Jones, P. T. et al. (1986) Nature 321:522-525; Riechmann, L. et al. (1988) 332:323-327; and Carter, P. et al. (1992) Proc. Natl. Acad. Sci. USA 89:4285-4289.

GnRH Compositions

Once the above GnRH polypeptides or antibodies are produced, they are formulated into compositions for delivery to a vertebrate subject. The relevant GnRH molecule is administered alone, or mixed with a pharmaceutically acceptable vehicle or excipient. Suitable vehicles are, for example, water, saline, dextrose, glycerol, ethanol, or the like, and combinations thereof. In addition, the vehicle may contain minor amounts of auxiliary substances such as wetting or emulsifying agents, pH buffering agents, or adjuvants in the case of vaccine compositions, which enhance the effectiveness of the vaccine. Suitable adjuvants are described further below. The compositions of the present invention can also include ancillary substances, such as pharmacological agents, cytokines, or other biological response modifiers.

As explained above, vaccine compositions of the present invention may include adjuvants to further increase the immunogenicity of the GnRH immunogen. Adjuvants may include for example, emulsifiers, muramyl dipeptides, avridine, aqueous adjuvants such as aluminum hydroxide and any of the various saponins, chitosan-based adjuvants, oils, and other substances known in the art. For example, compounds which may serve as emulsifiers herein include natural and synthetic emulsifying agents, as well as anionic, cationic and nonionic compounds. Among the synthetic compounds, anionic emulsifying agents include, for example, the potassium, sodium and ammonium salts of lauric and oleic acid, the calcium, magnesium and aluminum salts of fatty acids (i.e., metallic soaps), and organic sulfonates such as sodium lauryl sulfate. Synthetic cationic agents include, for example, cetyltrimethylammonium bromide, while synthetic nonionic agents are exemplified by glyceryl esters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The oil may be a mineral oil, a vegetable oil, or an animal oil. Mineral oil, or oil-in-water emulsions in which the oil component is mineral oil are preferred. In this regard, a “mineral oil” is defined herein as a mixture of liquid hydrocarbons obtained from petrolatum via a distillation technique; the term is synonymous with “liquid paraffin,” “liquid petrolatum” and “white mineral oil.” The term is also intended to include “light mineral oil,” i.e., an oil which is similarly obtained by distillation of petrolatum, but which has a slightly lower specific gravity than white mineral oil. See, e.g., Remington's Pharmaceutical Sciences, supra. A particularly preferred oil component is the oil-in-water emulsion sold under the trade name of EMULSIGEN PLUS™ (comprising a light mineral oil as well as 0.05% formalin, and 30 mcg/mL gentamicin as preservatives), available from MVP Laboratories, Ralston, Nebr. Suitable animal oils include, for example, cod liver oil, halibut oil, menhaden oil, orange roughy oil and shark liver oil, all of which are available commercially. Suitable vegetable oils, include, without limitation, canola oil, almond oil, cottonseed oil, corn oil, olive oil, peanut oil, safflower oil, sesame oil, soybean oil, and the like.

Alternatively, a number of aliphatic nitrogenous bases can be used as adjuvants with the vaccine formulations. For example, known immunologic adjuvants include amines, quaternary ammonium compounds, guanidines, benzamidines and thiouroniums (Gall, D. (1966) Immunology 11:369-386). Specific compounds include dimethyldioctadecylammonium bromide (DDA) (available from Kodak) and N,N-dioctadecyl-N,N-bis(2-hydroxyethyl)propanediamine (“avridine”). The use of DDA as an immunologic adjuvant has been described; see, e.g., the Kodak Laboratory Chemicals Bulletin 56(1):1-5 (1986); Adv. Drug Deliv. Rev. 5(3):163-187 (1990); J. Controlled Release 7:123-132 (1988); Clin. Exp. Immunol. 78(2):256-262 (1989); J. Immunol. Methods 97(2):159-164 (1987); Immunology 58(2):245-250 (1986); and Int. Arch. Allergy Appl. Immunol. 68(3):201-208 (1982). Avridine is also a well-known adjuvant. See, e.g., U.S. Pat. No. 4,310,550 to Wolff, III et al., which describes the use of N,N-higher alkyl-N′,N′-bis(2-hydroxyethyl)propane diamines in general, and avridine in particular, as vaccine adjuvants. U.S. Pat. No. 5,151,267 to Babiuk, and Babiuk et al. (1986) Virology 159:57-66, also relate to the use of avridine as a vaccine adjuvant.

Particularly preferred for use herein is an adjuvant known as “VSA-3” which is a modified form of the EMULSIGEN PLUS™ adjuvant which includes DDA (see, allowed U.S. patent application Ser. No. 08/463,837, incorporated herein by reference in its entirety).

Actual methods of preparing such dosage forms are known, or will be apparent, to those skilled in the art. See, e.g., Remington's Pharmaceutical Sciences, Mack Publishing Company, Easton, Pa., 18th edition, 1990. The composition or formulation to be administered will contain a quantity of the GnRH polypeptide adequate to achieve the desired state in the subject being treated.

The compositions of the present invention are normally prepared as injectables, either as liquid solutions or suspensions, or as solid forms which are suitable for solution or suspension in liquid vehicles prior to injection. The preparation may also be emulsified or the active ingredient encapsulated in liposome vehicles or other particulate carriers used.

The compositions may also be prepared in solid form. For example, solid particulate formulations can be prepared for delivery from commercially available needleless injector devices. Alternatively, solid dose implants can be provided for implantation into a subject. Controlled or sustained release formulations may also be used and are made by incorporating the GnRH polypeptides into carriers or vehicles such as liposomes, nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures.

Furthermore, the polypeptides may be formulated into compositions in either neutral or salt forms. Pharmaceutically acceptable salts include the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The composition is formulated to contain an effective amount of the GnRH polypeptide, the exact amount being readily determined by one skilled in the art, wherein the amount depends on the animal to be treated, in the case of a vaccine composition, the capacity of the animal's immune system to synthesize antibodies, and the degree of immunoneutralization of GnRH desired. For purposes of the present invention, formulations including approximately 1 μg to about 2 mg, more generally about 5 μg to about 800 μg, and even more particularly, 10 μg to about 400 μg of GnRH polypeptide per mL of injected solution should be adequate to raise an immunological response when administered. If a peptide-carrier chimera is used, the ratio of immunogen to carrier in the vaccine formulation will vary based on the particular carrier and immunogen selected to construct such molecules.

For example, if a leukotoxin-GnRH chimera is used, the ratio of GnRH to leukotoxin in the vaccine formulation will vary based on the particular leukotoxin and GnRH polypeptide moieties selected to construct those molecules. One preferred vaccine composition contains a leukotoxin-GnRH chimera having about 1 to 90% GnRH, preferably about 3 to 80% and most preferably about 10 to 70% GnRH polypeptide per fusion molecule. Increases in the percentage of GnRH present in the LKT-GnRH fusions reduce the amount of total antigen which must be administered to a subject in order to elicit a sufficient immunological response to GnRH.

The subject is administered one of the above-described compositions e.g., in a primary immunization, during the fattening period, in at least one dose, and optionally, two or more doses. The primary administration(s) is followed with one or more boosts with the same or different GnRH composition shortly before slaughter, in order to substantially reduce the circulating level of one or more androgenic and/or non-androgenic steroids.

Any suitable pharmaceutical delivery means may be employed to deliver the compositions to the vertebrate subject. For example, conventional needle syringes, spring or compressed gas (air) injectors (U.S. Pat. Nos. 1,605,763 to Smoot; 3,788,315 to Laurens; 3,853,125 to Clark et al.; 4,596,556 to Morrow et al.; and 5,062,830 to Dunlap), liquid jet injectors (U.S. Pat. Nos. 2,754,818 to Scherer; 3,330,276 to Gordon; and 4,518,385 to Lindmayer et al.), and particle injectors (U.S. Pat. Nos. 5,149,655 to McCabe et al. and 5,204,253 to Sanford et al.) are all appropriate for delivery of the compositions.

Preferably, the composition is administered intramuscularly, subcutaneously, intravenously, subdermally, intradermally, transdermally or transmucosally to the subject. If a jet injector is used, a single jet of the liquid vaccine composition is ejected under high pressure and velocity, e.g., 1200-1400 PSI, thereby creating an opening in the skin and penetrating to depths suitable for immunization.

Below are examples of specific embodiments for carrying out the present invention. The examples are offered for illustrative purposes only, and are not intended to limit the scope of the present invention in any way.

3. Experimental

### EXAMPLE 1

### Construction of pCB122 and pCB130

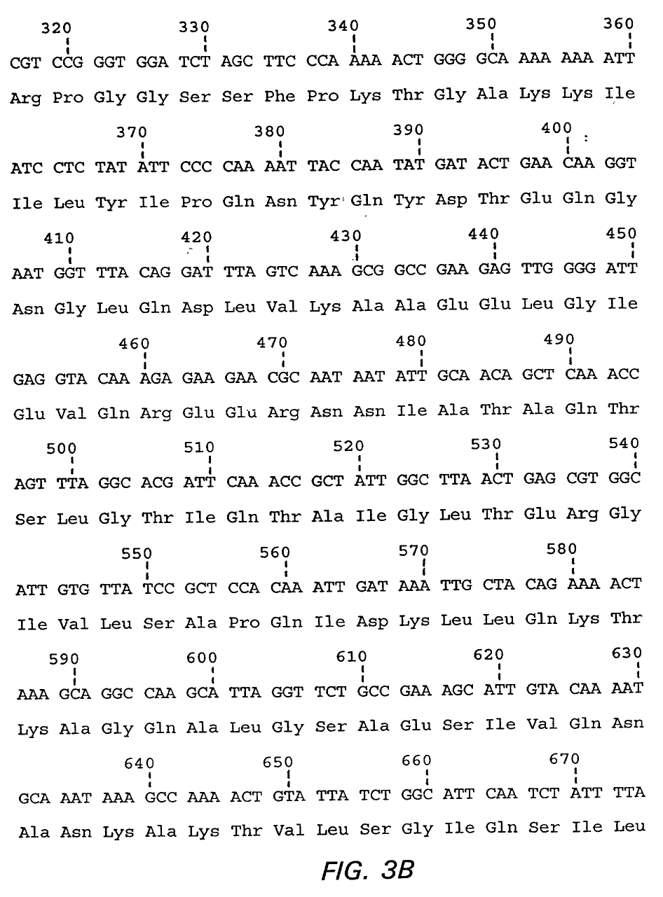

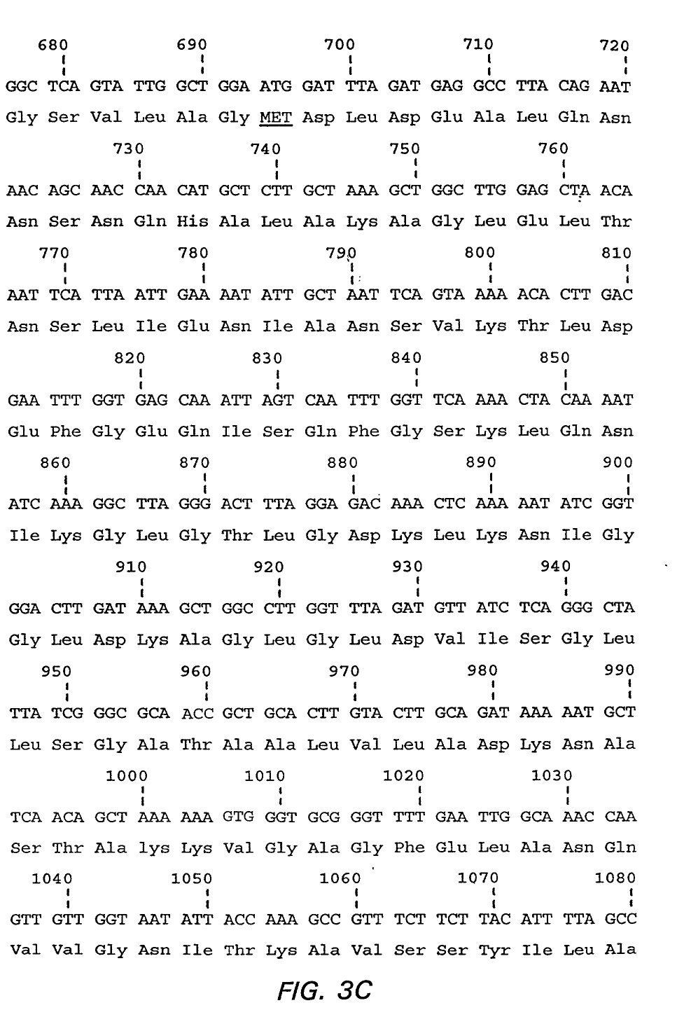

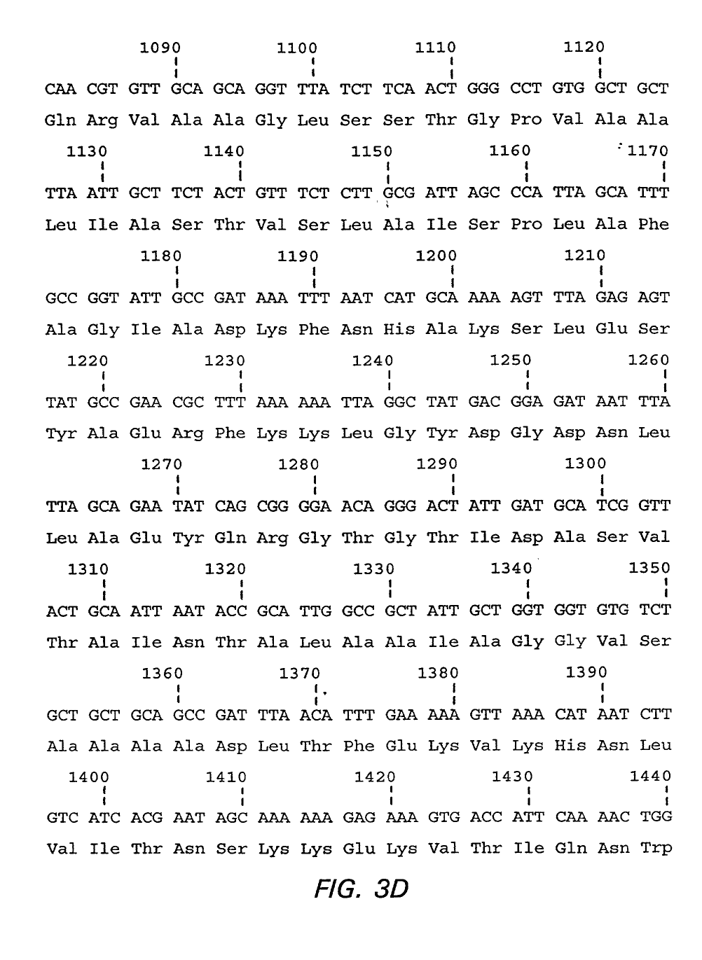

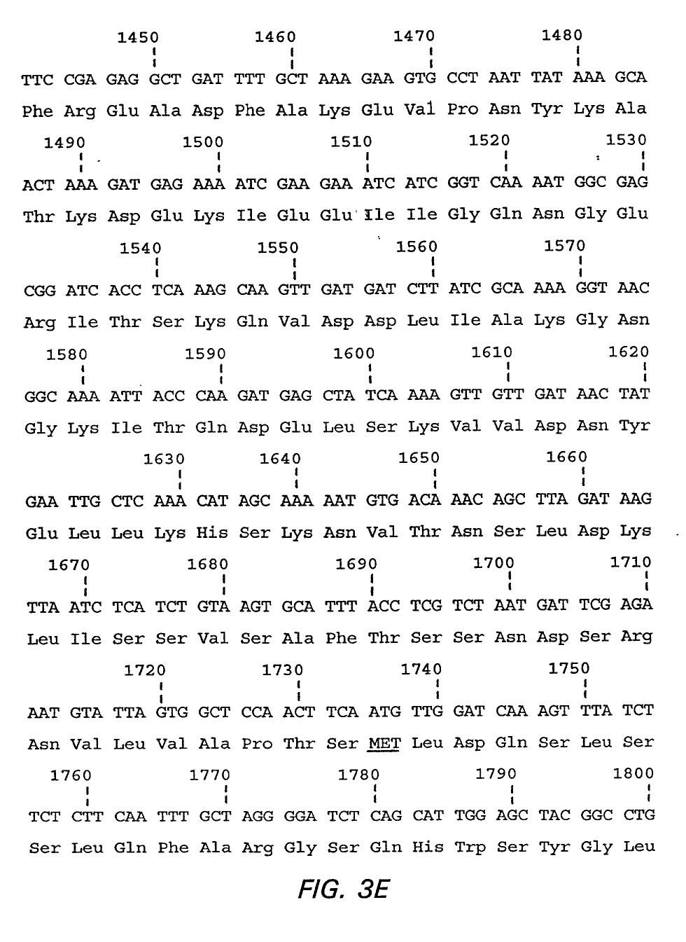

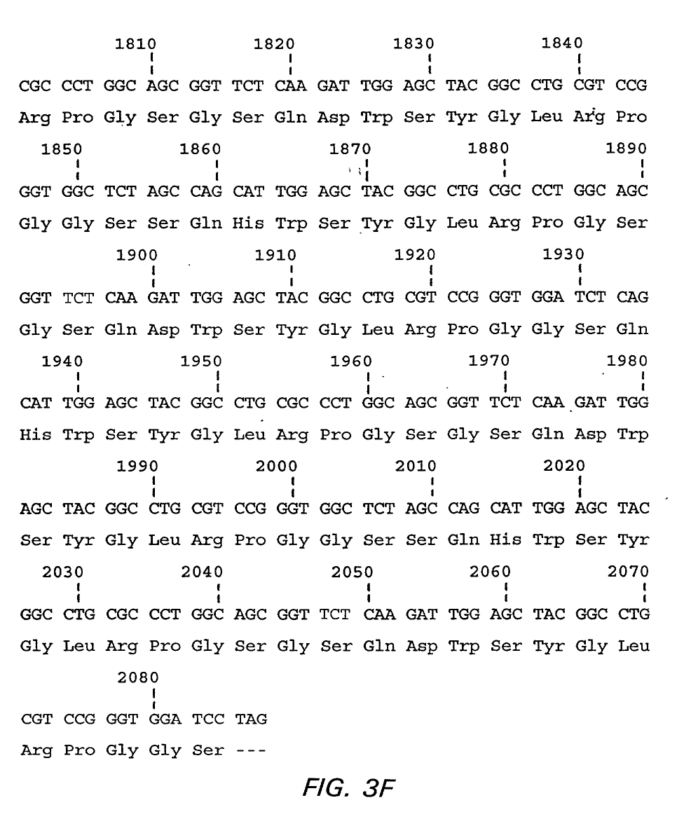

Plasmids pCB122 and pCB130 were used to produce a GnRH fusion protein for use in the examples described below. Both plasmids produce a protein with the same amino acid sequence. The GnRH construct in both plasmids contains 8 tandem repeats of the GnRH sequence fused to both the 5′ and 3′ ends of a DNA sequence coding for a carrier leukotoxin polypeptide. Each alternating GnRH sequence has a change in the fourth base in the sequence from cytosine to guanosine. This results in a single amino acid change in the second amino acid of the GnRH molecule from His to Asp. See, FIGS. 2A and 2B. The leukotoxin portion of the construct encodes a shortened version of leukotoxin which was developed from the recombinant leukotoxin gene present in plasmid pAA352 (ATCC Accession No. 68283 and described in U.S. Pat. No. 5,476,657, incorporated herein by reference in its entirety) by removal of an internal DNA fragment of approximately 1300 bp in length. The leukotoxin polypeptide has an estimated molecular weight of 52 kDa and contains convenient restriction sites for use in producing the fusion proteins of the present invention. The chimeric construct is under the control of the Tac promoter and induction is controlled through the use of Lac I. The GnRH-leukotoxin fusion protein produced by plasmids pCB122 and pCB130 is shown in FIGS. 3A through 3F.

Plasmid pCB122 was prepared as follows. The leukotoxin gene was isolated as described in U.S. Pat. Nos. 5,476,657 and 5,837,268, incorporated herein by reference in their entireties. In particular, to isolate the leukotoxin gene, gene libraries of P. haemolytica A1 (strain B122) were constructed using standard techniques. See, Lo et al., Infect. Immun., supra; DNA CLONING: Vols. I and II, supra; and Sambrook et al., supra. A genomic library was constructed in the plasmid vector pUC13 and a DNA library constructed in the bacteriophage lambda gt11. The resulting clones were used to transform E. coli and individual colonies were pooled and screened for reaction with serum from a calf which had survived a P. haemolytica infection and that had been boosted with a concentrated culture supernatant of P. haemolytica to increase anti-leukotoxin antibody levels. Positive colonies were screened for their ability to produce leukotoxin by incubating cell lysates with bovine neutrophils and subsequently measuring release of lactate dehydrogenase from the latter.

Several positive colonies were identified and these recombinants were analyzed by restriction endonuclease mapping. One clone appeared to be identical to a leukotoxin gene cloned previously. See, Lo et al., Infect. Immun., supra. To confirm this, smaller fragments were re-cloned and the restriction maps compared. It was determined that approximately 4 kilobase pairs of DNA had been cloned. Progressively larger clones were isolated by carrying out a chromosome walk (5′ to 3′ direction) in order to isolate full-length recombinants which were approximately 8 kb in length. The final construct was termed pAA114. This construct contained the entire leukotoxin gene sequence.

lktA, a MaeI restriction endonuclease fragment from pAA114 which contained the entire leukotoxin gene, was treated with the Klenow fragment of DNA polymerase I plus nucleotide triphosphates and ligated into the SmaI site of the cloning vector pUC13. This plasmid was named pAA179. From this, two expression constructs were made in the ptac-based vector pGH432:lacI digested with SmaI. One, pAA342, consisted of the 5′-AhaIII fragment of the lktA gene while the other, pAA345, contained the entire MaeI fragment described above. The clone pAA342 expressed a truncated leukotoxin peptide at high levels while pAA345 expressed full length leukotoxin at very low levels. Therefore, the 3′ end of the lktA gene (StyI BamHI fragment from pAA345) was ligated to StyI BamHI-digested pAA342, yielding the plasmid pAA352. The P. haemolytica leukotoxin produced from the pAA352 construct is hereinafter referred to as LKT 352.

Plasmid pAA352 was then used to prepare a shortened version of the recombinant leukotoxin polypeptide. The shortened LKT gene was produced by deleting an internal DNA fragment of approximately 1300 bp in length from the recombinant LKT gene as follows. The plasmid pCB113, (ATCC Accession No. 69749 and described in U.S. Pat. No. 5,837,268, incorporated herein by reference in its entirety) which includes the LKT 352 polypeptide, was digested with the restriction enzyme BstB1 (New England Biolabs). The resultant linearized plasmid was then digested with mung-bean nuclease (Pharmacia) to remove the single stranded protruding termini produced by the BstB1 digestion. The blunted DNA was then digested with the restriction enzyme Nae1 (New England Biolabs), and the digested DNA was loaded onto a 1% agarose gel where the DNA fragments were separated by electrophoresis. A large DNA fragment of approximately 6190 bp was isolated and purified from the agarose gel using a Gene Clean kit (Bio 101), and the purified fragment was allowed to ligate to itself using bacteriophage T4 DNA ligase (Pharmacia). The resulting ligation mix was used to transform competent E. coli JM105 cells, and positive clones were identified by their ability to produce an aggregate protein having an appropriate molecular weight. The recombinant plasmid thus formed was designated pCB111, (ATCC Accession No. 69748), and produces a shortened leukotoxin polypeptide (hereinafter referred to as LKT 111) fused to four copies of GnRH polypeptide. Plasmid pCB114 has the multiple copy GnRH sequence (corresponding to the oligomer of FIG. 2B) inserted twice. Both these plasmids are described in U.S. Pat. No. 5,837,268, incorporated herein by reference in its entirety, and produce shortened leukotoxin polypeptides termed LKT 111 and LKT 114, respectively.

A recombinant LKT-GnRH fusion molecule having two 8 copy GnRH multimers, one arranged at the N′-terminus of LKT 114 and the other arranged at the C′-terminus of LKT 114, was constructed from the LKT-GnRH fusion sequence obtained from the pCB114 plasmid by ligating the multiple copy GnRH sequence (corresponding to the oligomer of FIG. 2B) twice at the 5′ end of the LKT 114 coding sequence. A synthetic nucleic acid molecule having the following nucleotide sequence: 5′-ATGGCTACTGTTATAGATCGATCT-3′ (SEQ ID NO:______) was ligated at the 5′ end of the multiple copy GnRH sequences. The synthetic nucleic acid molecule encodes an eight amino acid sequence (Met-Ala-Thr-Val-Ile-Asp-Arg-Ser) (SEQ ID NO:______). The resulting recombinant molecule thus contains in the order given in the 5′ to 3′ direction: the synthetic nucleic acid molecule; a nucleotide sequence encoding a first 8 copy GnRH multimer; a nucleotide sequence encoding the shortened LKT peptide (LKT 114); and a nucleotide sequence encoding a second 8 copy GnRH multimer.

The recombinant molecule was circularized, and the resulting molecule was used to transform competent E. coli JM105 cells. Positive clones were identified by their ability to produce an aggregate protein having a molecular weight of approximately 74 KDa. The recombinant plasmid thus formed was designated pCB122 which produces the LKT 114 polypeptide fused to 16 copies of GnRH polypeptide.

For plasmid pCB130, the amp^{r} gene or pCB122 was replaced with the tet^{r} gene. Thus, the plasmid is under tetracycline selection. The nucleotide sequence of the recombinant LKT-GnRH fusion of plasmids pCB122 and pCB130 is shown in FIGS. 3A through 3F.

### EXAMPLE 2

### Purification of LKT-antigen Fusions

The recombinant LKT-GnRH fusion from Example 1 was purified using the following procedure. Five to ten colonies of transformed E. coli strains were inoculated into 10 mL of TB broth supplemented with 100 μg/mL of ampicillin and incubated at 37° C. for 6 hours on a G10 shaker, 220 rpm. Four mL of this culture was diluted into each of two baffled Fernbach flasks containing 400 mL of TB broth+ampicillin and incubated overnight as described above. Cells were harvested by centrifugation for 10 minutes at 4,000 rpm in polypropylene bottles, 500 mL volume, using a Sorvall GS3 rotor. The pellet was resuspended in an equal volume of TB broth containing ampicillin which had been prewarmed to 37° C. (i.e., 2×400 ml), and the cells were incubated for 2 hours as described above.

3.2 mL of isopropyl-B,D-thiogalactopyranoside (IPTG, Gibco/BRL), 500 mM in water (final concentration=4 mM), was added to each culture in order to induce synthesis of the recombinant fusion proteins. Cultures were incubated for two hours. Cells were harvested by centrifugation as described above, resuspended in 30 mL of 50 mM Tris-hydrochloride, 25% (w/v) sucrose, pH 8.0, and frozen at −70° C. The frozen cells were thawed at room temperature after 60 minutes at −70° C., and 5 mL of lysozyme (Sigma, 20 mg/mL in 250 mM Tris-HCl, pH 8.0) was added. The mixture was vortexed at high speed for 10 seconds and then placed on ice for 15 minutes. The cells were then added to 500 mL of lysis buffer in a 1000 mL beaker and mixed by stirring with a 2 mL pipette. The beaker containing the lysed cell suspension was placed on ice and sonicated for a total of 2.5 minutes (5-30 second bursts with 1 minute cooling between each) with a Braun sonicator, large probe, set at 100 watts power. Equal volumes of the solution were placed in Teflon SS34 centrifuge tubes and centrifuged for 20 minutes at 10,000 rpm in a Sorvall SS34 rotor. The pellets were resuspended in a total of 100 mL of sterile double distilled water by vortexing at high speed, and the centrifugation step repeated. Supernatants were discarded and the pellets combined in 20 mL of 10 mM Tris-HCl, 150 mM NaCl, pH 8.0 (Tris-buffered saline) and the suspension frozen overnight at −20° C.

The recombinant suspension was thawed at room temperature and added to 100 mL of 8 M Guanidine HCl (Sigma) in Tris-buffered saline and mixed vigorously. A magnetic stir bar was placed in the bottle and the solubilized sample was mixed at room temperature for 30 minutes. The solution was transferred to a 2000 mL Erlenmeyer flask and 1200 mL of Tris-buffered saline was added quickly. This mixture was stirred at room temperature for an additional 2 hours. 500 mL aliquots were placed in dialysis bags (Spectrum, 63.7 mm diameter, 6,000-8,000 MW cutoff, #132670, from Fisher scientific) and these were placed in 4,000 mL beakers containing 3,500 mL of Tris-buffered saline+0.5 M Guanidine HCl. The beakers were placed in a 4° C. room on a magnetic stirrer overnight after which dialysis buffer was replaced with Tris-buffered saline+0.1 M Guanidine HCl and dialysis continued for 12 hours. The buffer was then replaced with Tris-buffered saline+0.05 M Guanidine HCl and dialysis continued overnight. The buffer was replaced with Tris-buffered saline (no guanidine), and dialysis continued for 12 hours. This was repeated three more times. The final solution was poured into a 2000 mL plastic roller bottle (Corning) and 13 mL of 100 mM PMSF (in ethanol) was added to inhibit protease activity. The solution was stored at −20° C. in 100 mL aliquots.

To confirm that the fusion protein had been isolated, aliquots of each preparation were diluted 20-fold in double distilled water, mixed with an equal volume of SDS-PAGE sample buffer, placed in a boiling water bath for five minutes and run through 12% polyacrylamide gels. Recombinant leukotoxin controls were also run. The fusion protein was expressed at high levels as inclusion bodies.

### EXAMPLE 3

### Antibody Titers Following GnRH Immunization on Pigs

This trial was designed to evaluate variables including volume, site of the second injection relative to the first and the number (one vs two) injections for the primary vaccination. For the study, 160 pigs, 28 days of age and weighing 3 to 4 kg, were assigned to one of eight treatment groups (see Table 1). There were 10 female and 10 castrate male pigs in each group. Animals were housed 10 per pen and were cared for using Standard Operating Procedures developed by the Prairie Swine Centre, Inc., an experimental facility affiliated with the University of Saskatchewan and inspected by the Canadian Council on Animal Care.

For Groups 1 to 7, GnRH vaccines were made using the GnRH immunogen from plasmid pCB122, described above. In particular, the GnRH immunogen was dissolved at a concentration of 20 mg/mL in 8 M urea. The adjuvant used to formulate the GnRH vaccines was VSA-3, a modified form of the EMULSIGEN PLUS™ adjuvant which includes DDA (see, allowed U.S. patent application Ser. No. 08/463,837, incorporated herein by reference in its entirety).

The GnRH vaccines were prepared by combining the stock solution of GnRH immunogen with phosphate buffered saline and mixing with VSA-3 at a ratio of 1:1 (v/v) to form a stable emulsion. The dose of GnRH immunogen for Groups 1, 2, 3, 4, and 6 was 40 μg, however the volume differed in some of the formulations. Table 1 provides details for each treatment Group. The GnRH vaccines for Groups 5 and 7 contained 30 μg of the GnRH immunogen/0.25 mL while Group 8 received 40 μg of the GnRH immunogen from plasmid pCB130 in 0.4 mL of adjuvant. In all instances the ratio of VSA-3 adjuvant to the aqueous phase (phosphate buffered saline) remained at 1:1 (v/v). Adjustments were made by altering the volume of stock immunogen solution.

TABLE 1
Dose, site and volume of GnRH vaccine
administered at the first and second injections

| | | Dose | First | Volume (mL) | Second | Volume (mL) |
|---|---|---|---|---|---|---|
| Group | N | (μg) | Injection | First Injection | Injection | Second Injection |
| 1 | 20 | 40 | L* | 0.15 | R* | 0.15 |
| 2 | 20 | 40 | L | 0.25 | R | 0.25 |
| 3 | 20 | 40 | L | 0.35 | R | 0.35 |
| 4 | 20 | 40 | L | 0.25 | L | 0.25 |
| 5 | 20 | 60 | L + R | 0.25** | R | 0.25 |
| 6 | 20 | 40 | L + R | 0.15** | R | 0.30 |
| 7 | 20 | 60 | L + R | 0.25** | Neck | 0.25 |
| 8 | 20 | 40 | L | 0.25 | R | 0.25 |

*L and R refer to Left and Right ear.
**The volume indicated was given in each of the ears. Therefore, Groups 5, 6 and 7 received a total of 0.5, 0.3 and 0.5 mL respectively at the primary injection.

The vaccines were all administered with a Biojector 2000 needleless injection device manufactured by Bioject Inc., Portland, Oreg., USA. This device utilizes a gas cylinder to inject the vaccine under high pressure through a small opening. The vaccine penetrates through the skin and is deposited subcutaneously. In each treatment group, the first injection was given when the pigs were 28 days old and the second was given 35 days later.

Injections were given on the outer surface of the pinna of the ear except for the second injection in Group 7 which was given on the dorsal midline 10-15 cm behind the head. Blood was collected by jugular veinpuncture at Days 35, 49 and 63 of the trial (relative to the beginning of the study (Day 0)). Blood was allowed to clot at room temperature and then was centrifuged to harvest serum which was stored at −20° C. until it was analyzed for GnRH antibody titers.

GnRH antibody titers were determined by a modified radioimmunoassay procedure. Synthetic GnRH (Bachem, Inc.) was iodinated with I¹²⁵ (Amersham, Oakville, Ontario). Dilutions of serum were added to tet tubes followed by a standard amount of I¹²⁵ labeled GnRH to give a final incubation volume of 0.7 mL. A suspension of charcoal in assay buffer was added at the end of a 24 hour incubation at 2-6° C. to absorb the non-antibody bound I¹²⁵-GnRH. After centrifugation, radioactivity in the charcoal fraction was measured. Data are presented as a numeric value which is the % of a standard dose (approximately 12,000 cpm) of I¹²⁵-GnRH bound to antibody at a specific serum dilution.

Descriptive statistics, analysis of variance and “t” tests were done using the Student Version of Statistix, Version 1, Copyright 1996.

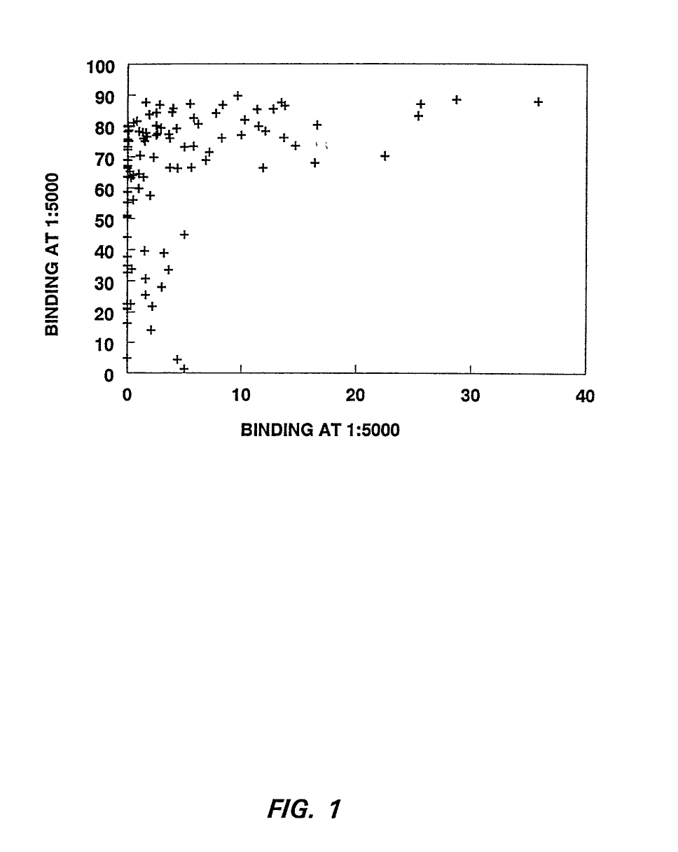

Volumes of 0.15, 0.25 and 0.35 given at a single injection site were evaluated in Groups 1, 2 and 3, respectively. FIG. 1 shows the relationship between antibody titer before the booster vaccination on Day 35 of the trial, when animals were 63 days of age, and 14 days after booster injection, Day 49 of the trial when animals were 77 days of age. Animals that had titers greater than 10% binding at 1:5000 on Day 35 gave a better response to the booster vaccination than animals that had a weaker response to the primary injection. Based on other experiments, we know that binding of approximately 20% at a 1:5000 dilution will give partial suppression of testosterone secretion.

These results indicate the utility of a strong response to the primary immunization providing there is no effect on growth or efficiency of feed utilization.

### EXAMPLE 4

### Effects of GnRH Immunization on Testosterone Levels

The following experiment utilizes an immunological approach to demonstrate the lack of effect of reducing testosterone concentrations in prepubertal animals. Sixty intact male pigs were divided into 3 treatment groups. Group 1 was castrated surgically at birth and Groups 2 and 3 were left intact. At approximately 21 days of age, Group 3 was immunized with a GnRH immunogen comprising eight copies of GnRH linked to an internally deleted leukotoxin molecule comprising amino acids 38-378 and 815-951 of native leukotoxin. The GnRH immunogen was formulated in VSA-3 adjuvant as described above. The immunization resulted in an increase in antibody production sufficient to cause a detectable decrease in testosterone secretion. Group 2 was left intact throughout the experiment and was not immunized.

Previous studies have shown that animals immunized with this adjuvant will have a moderate, sustained increase in GnRH antibody titers which reduces testosterone concentrations to low but detectable levels. Feed consumption and carcass composition were measured during the experiment to compare those parameters at various ages.

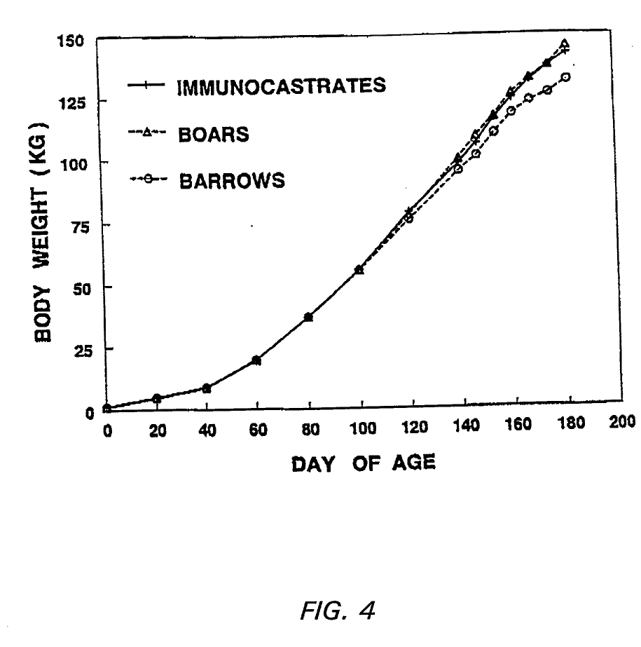

Animals treated with this GnRH vaccine (immunocastrates) performed similarly to the castrated males (barrows) and uncastrated males (boars) until approximately 90 days of age. Furthermore, as shown in FIG. 4, all three groups had similar body weight gain.

### EXAMPLE 5

### Immunocastration of Sexually Mature Pigs by GnRH Vaccination

The objects of this study were to determine if GnRH vaccination decreased serum testosterone and fat androstenone concentrations in sexually mature male pigs to values equivalent to those seen in surgically castrated pigs and to determine the kinetics of GnRH antibody response, serum testosterone concentrations and fat androstenone levels after a primary and secondary immunization.

24 intact male pigs were assigned randomly prior to Day 0 to one of three treatment groups (Groups 1, 2 and 3) as shown in Table 2. Six age—and litter-matched pigs which had been surgically castrated at less than 1 week of age were assigned to a fourth treatment group (Group 4—early castrates). Pigs were housed 10 animals per pen until they were approximately 60 kg in weight at which time they were housed 2 animals per pen. Pigs were provided free access to feed and water and were cared for using standard operating procedures documented by the Prairie Swine Centre, an animal facility affiliated with the University of Saskatchewan and inspected by the Canadian Council on Animal Care.

GnRH vaccines were made using the GnRH immunogen from plasmid pCB122, dissolved at a concentration of 28 mg/ml in 4M guanidine HCL. The adjuvant used to formulate the GnRH vaccine was VSA-3. The vaccine was prepared by combining the GnRH immunogen with phosphate buffered saline and mixing with VSA-3 at a ratio of 1:1 (v/v) to form a stable emulsion. The vaccine contained 40 μg GnRH immunogen per 0.5 ml dose and was administered IM. The placebo contained phosphate buffered saline and VSA-3.

Pigs were given two IM injections of vaccine or placebo in the neck. The first injection was given at Day 0 of the experiment at which time the pigs were 21 days of age. The second injection was given when the pigs were approaching sexual maturity at which time they were approximately 100 kg of body weight (Day 110-Day 120) (Table 2). Pigs in Group 2 (late castrates) were castrated surgically when they reached sexual maturity which is influenced strongly by body weight and occurs at approximately 110 kg in body weight (Day 115 to 125). Pigs in Group 1 received the second immunization approximately 1 week prior to when the pigs in Group 2 were castrated surgically. This was done in order to allow the GnRH antibody titers generated by the second immunization to reach biologically effective levels at approximately the same time that the animals in Group 2 were surgically castrated.

TABLE 2
Description of number of animals, treatment and time of
surgical or immunological castration

| | | | Vaccine or | Time of Surgical or |
|---|---|---|---|---|
| Gr # | n | Treatment | Placebo | Immunological Castration |
| 1 | 10 | Immunized | Vaccine | 100 kg (approx. Day 120) |
| 2 | 6 | Late | Placebo | 110 kg (approx. Day 120) |
| | | Castrates | | |
| 3 | 8 | Intact Males | Placebo | Not Done |
| 4 | 6 | Early | Placebo | 2-3 kg |
| | | Castrates | | (<1 week of age) |

In order to simplify data analysis and presentation, Day 120 was referred to as the time of the “events”, i.e., when the animals received either the second injection (all groups) or were surgically castrated (Group 2). All data collected subsequent to the “events” are described relative to the “events”, i.e. 7 days after the “events” is referred to as Day 127, 14 days after the “events” is referred to as Day 134, etc.

Blood samples were obtained from all pigs by jugular veinpuncture at approximately 28 day intervals between Days 28 and 120. Thereafter, blood was obtained at weekly intervals from all pigs until animals were killed on Day 162 of the experiment (42 days after the “events”). Blood was allowed to clot at room temperature, centrifuged and the serum was frozen within 24 hours after sampling.

Individual weight gains were determined monthly by weighing all animals from Day 0 until the “events” after which time they were weighed weekly until slaughter.

Subcutaneous fat samples (approximately 5 g) for androstenone measurements were obtained under local anesthesia from alternate sides of the neck of all pigs at the time of the “events” and at weekly intervals until slaughter (Day 162). Fat samples were chilled immediately and frozen within 4 hours after biopsy.

Measurements at the time of slaughter included carcass weight, backfat depth at the level of the 10th rib, testicular weight and bulbo-urethral gland length.

GnRH antibody titers were determined by a modified radioimmunoassay procedure. Synthetic GnRH (Bachem, Inc.) was iodinated with I¹²⁵ (Amersham, Oakville, Ontario). Dilutions of serum were added to test tubes followed by a standard amount of I¹²⁵-labeled GnRH to give a final incubation volume of 0.7 ml. A suspension of charcoal in assay buffer was added at the end of a 24 hour incubation at 2-6° C. to adsorb the non-antibody bound I¹²⁵-GnRH. After centrifugation, radioactivity in the charcoal fraction was measured. data was presented as a numeric value which is the % of a standard dose (approximately 12,000 cpm) of I¹²⁵-GnRH bound to antibody at a specified serum dilution.

Serum testosterone was measured using a Coat-A-Count total testosterone kit (DPS, Los Angeles, Calif.). this assay is based on I¹²⁵-testosterone and antibodies that have a high specificity of testosterone.

Fat androstenone concentrations were determined using a colorimetric method.

Primary outcome measurements included GnRH antibody titers measured as % binding at a serum dilution of 1:5000 in Group 1, and at a serum dilution of 1:100 in Groups 2, 3 and 4. Serum testosterone concentrations, fat androstenone, body weight, backfat, testicular weight and bulbourethrethral length were also measured.

All pigs in Group 1 developed GnRH antibody titers that were readily detectable at 1:100 serum dilution after primary immunization (see Table 3). Furthermore, immunization of pigs at 21 and approximately 140 days of age generated GnRH antibody titers which resulted in a decline in serum testosterone and fat androstenone concentrations equivalent to those seen in pigs castrated early and late in life. A significant reduction in the size of the testes and bulbourethral glands was also seen in immunized pigs, as compared to intact males.

TABLE 3
Kinetics of anti-GnRH antibody titers in Group 1 in male pigs
after a primary immunization (1:100 dilution)

| | Anti-GnRH antibody titer 1:100 dilution (days after primary | | | | |
|---|---|---|---|---|---|
| | immunization) | | | | |
| Animal | Day 28 | Day 56 | Day 84 | Day 112 | Day 116 |
| 1 | 22.90 | 31.40 | 13.70 | 11.80 | 37.00 |
| 5 | 0.60 | 21.50 | 17.00 | 11.40 | 9.20 |
| 6 | 61.50 | 52.40 | 27.90 | 15.10 | 14.20 |
| 9 | 28.10 | 19.00 | 7.90 | 5.30 | 36.80 |
| 10 | 75.50 | 77.60 | 75.10 | 71.40 | 74.40 |
| 13 | 4.20 | 4.70 | 2.60 | 6.10 | 18.40 |
| 14 | 3.60 | 22.90 | 30.90 | 30.10 | 57.40 |
| 17 | 34.70 | 50.70 | 57.00 | 56.00 | 52.60 |
| 18 | 42.10 | 36.40 | 28.30 | 14.40 | 14.90 |
| 21 | 39.50 | 39.60 | 27.10 | 19.50 | 15.40 |
| 22 | 38.00 | 60.10 | 51.00 | 37.50 | 72.70 |

### EXAMPLE 6

### GnRH Immunization of Bulls

This experiment was conducted with 58 prepubertal bull calves. Twenty-eight bull calves in Group I were vaccinated subcutaneously twice with a vaccine composition comprising 200 μg of the GnRH immunogen derived from plasmid pCB122 in VSA-3 adjuvant (Day 0 and Day 56) and 30 control bulls in Group 2 were vaccinated with a placebo. Vaccinations of Group I resulted in significant titers against GnRH by Day 42, significant reductions in scrotal circumferences by Day 84 and significantly reduced testosterone levels by Day 98 (Table 4). Despite these significant anti-GnRH titers and reduced testosterone, no differences in daily gain or feed efficiency were observed in the period from Day 0 to 84 (Table 5).

TABLE 4
Effect of GnRH vaccine on anti-GnRH titers, scrotal circumference and
serum testosterone in bull calves in Groups 1 and 2.

| | GnRH | | Serum Testosterone | | Scrotal Circumference | |
|---|---|---|---|---|---|---|
| Day of | Titers* | | (ng/ml) | | (cm) | |
| Experiment | Grp 1 | Grp 2 | Grp 1 | Grp 2 | Grp 1 | Grp 2 |
| 0 | 3.3 | 3.8 | 2.7 | 3.7 | 23.0 | 23.3 |
| 14 | 3.5 | 0.5 | | | | |
| 27 | 2.7 | 0.6 | | | 26.6 | 26.0 |
| 42 | 6.6^{a} | 0.4^{b} | | | | |
| 56 | 10.4^{a} | 1.6^{a} | | | 28.0 | 28.3 |
| 70 | 62.1^{a} | 1.2^{b} | 5.8 | 5.2 | | |
| 84 | 63.1^{a} | 2.1^{b} | | | 28.4^{a} | 29.8^{b} |
| 98 | 55.8^{a} | 8.7^{b} | 2.9^{a} | 7.7^{b} | 29.2^{a} | 30.9^{b} |
| 112 | 50.7^{a} | 5.3^{b} | | | | |
| 126 | | | 5.4^{a} | 9.1^{b} | 29.3^{a} | 32.2^{b} |

*Measured as % binding of GnRH-I⁻¹²⁵ at a 1:1,000 serum dilution
Statistical comparisons were made between GnRH immunized and control groups.
Values with different superscripts (a vs. b) differ (p < 05).

TABLE 5
Effect of GnRH vaccine on average daily gain, feed intake and
feed efficiency from Day 0 to 84. Analysis of variance indicated there
were no statistical differences among any parameters measured.

| | Group 1 | Group 2 |
|---|---|---|
| Variable | GnRH Immunized | Control |
| Daily Gain (kg/day) | 1.24 | 1.24 |
| Feed Intake (kgDM/day) | 7.32 | 7.50 |
| Feed Efficiency (kgDM/kg gain) | 5.90 | 6.06 |

These novel findings indicate there is an important utility for a GnRH vaccine which gives a sufficiently strong immune response after the primary immunization to result in antibody titers which give a detectable reduction in serum testosterone but which does not significantly reduce growth or feed efficiency. These findings are particularly novel because they show that temporary suppression of androgen secretion during the early growth period does not suppress body growth or feed efficiency. This has particular utility when using vaccination protocols which require subsequent immunization later in life with the objective of achieving a strong secondary response.

Deposits of Strains Useful in Practicing the Invention

A deposit of biologically pure cultures of the following strains was made with the American Type Culture Collection (ATCC), 10801 University Boulevard, Manassas, Va. The accession number indicated was assigned after successful viability testing, and the requisite fees were paid. The deposits were made under the provisions of the Budapest Treaty on the International Recognition of the Deposit of Microorganisms for the Purpose of Patent Procedure and the Regulations thereunder (Budapest Treaty). This assures maintenance of viable cultures for a period of thirty (30) years from the date of deposit and at least five (5) years after the most recent request for the furnishing of a sample of the deposit by the depository. The organisms will be made available by the ATCC under the terms of the Budapest Treaty, which assures permanent and unrestricted availability of the cultures to one determined by the U.S. Commissioner of Patents and Trademarks to be entitled thereto according to 35 U.S.C. §122 and the Commissioner's rules pursuant thereto (including 37 C.F.R. §1.12). Upon the granting of a patent, all restrictions on the availability to the public of the deposited cultures will be irrevocably removed.

These deposits are provided merely as convenience to those of skill in the art, and are not an admission that a deposit is required under 35 U.S.C. §112. The nucleic acid sequences of these plasmids, as well as the amino acid sequences of the polypeptides encoded thereby, are incorporated herein by reference and are controlling in the event of any conflict with the description herein. A license may be required to make, use, or sell the deposited materials, and no such license is hereby granted.

| Strain | Deposit Date | ATCC No. |
|---|---|---|
| pAA352 in E. coli W1485 | Mar. 30, 1990 | 68283 |
| pCB113 in E. coli JM105 | Feb. 1, 1995 | 69749 |
| pCB111 in E. coli JM105 | Feb. 1, 1995 | 69748 |
| pCB130 in | | |

Thus, methods of immunizing against GnRH are disclosed. Although preferred embodiments of the subject invention have been described in some detail, it is understood that obvious variations can be made without departing from the spirit and the scope of the invention as defined by the appended claims.

We claim:

1. A method of raising an uncastrated male food-producing animal for meat production comprising vaccinating said animal with a first vaccine composition comprising a GnRH immunogen prior to or during the fattening period of said animal to cause a reduction in circulating testosterone levels, and vaccinating said animal with a second vaccine composition comprising a GnRH immunogen at about 2 to about 8 weeks before slaughter of the animal to substantially reduce the level of one or more androgenic and/or non-androgenic steroids.

2. The method of claim 1, wherein said first vaccine composition is administered to said animal at a time between the birth of the animal and about 10 weeks of age.

3. The method of claim 1, wherein the first and second vaccine compositions further comprise an immunological adjuvant.

4. The method of claim 3, wherein the immunological adjuvant in said first vaccine composition comprises an oil and dimethyldioctadecylammonium bromide.

5. The method of claim 3, wherein the immunological adjuvant in said second vaccine composition comprises an oil and dimethyldioctadecylammonium bromide.

6. The method of claim 3, wherein the GnRH immunogen in the first and second vaccine compositions is the same.

7. The method of claim 3, wherein the GnRH immunogen in the first and second vaccine compositions is different.

8. The method of claim 1, wherein administration of the first vaccine composition results in the production of antibodies that cross-react with endogenous GnRH of said animal and the second composition is administered after the antibody levels have declined.

9. The method of claim 3, wherein said GnRH immunogen in said first vaccine composition is a GnRH multimer comprising the general formula (GnRH-X-GnRH)y wherein:
GnRH is a GnRH immunogen;
X is one or more molecules selected from the group consisting of a peptide linkage, an amino acid spacer group, a carrier molecule and [GnRH]ₙ, where n is an integer greater than or equal to 1; and
y is an integer greater than or equal to 1.

10. The method of claim 3, wherein said GnRH immunogen in said first and second vaccine compositions is a GnRH multimer comprising the general formula (GnRH-X-GnRH)y wherein:
GnRH is a GnRH immunogen;
X is one or more molecules selected from the group consisting of a peptide linkage, an amino acid spacer group, a carrier molecule and [GnRH]ₙ, where n is an integer greater than or equal to 1; and
y is an integer greater than or equal to 1.

11. The method of claim 9, wherein the carrier molecule is a leukotoxin polypeptide.

12. The method of claim 10, wherein the carrier molecule is a leukotoxin polypeptide.

13. The method of claim 1, wherein said GnRH immunogen is a nucleic acid molecule.

14. The method of claim 1, wherein the second vaccine composition is administered at about 4 to about 6 weeks before slaughter of the animal.

15. The method of claim 14, wherein the immunological adjuvant in said second vaccine composition is an aqueous adjuvant.

16. A method of raising an uncastrated male bovine, ovine or porcine animal for meat production comprising vaccinating said animal with a first vaccine composition comprising a GnRH immunogen prior to or during the fattening period of said animal to cause a reduction in circulating testosterone levels, and vaccinating said animal with a second vaccine composition comprising a GnRH immunogen at about 2 to about 8 weeks before slaughter of the animal, to substantially reduce the level of one or more androgenic and/or non-androgenic steroids.

17. The method of claim 16, wherein the first and second vaccine compositions further comprise an immunological adjuvant.

18. The method of claim 17, wherein the GnRH immunogen in the first and second vaccine compositions is the same.

19. The method of claim 17, wherein the GnRH immunogen in the first and second vaccine compositions is different.

20. The method of claim 16, wherein said GnRH immunogen in said first vaccine composition is a GnRH multimer comprising the general formula (GnRH-X-GnRH)y wherein:
GnRH is a GnRH immunogen;
X is one or more molecules selected from the group consisting of a peptide linkage, an amino acid spacer group, a carrier molecule and [GnRH]ₙ, where n is an integer greater than or equal to 1; and
y is an integer greater than or equal to 1.

21. The method of claim 18, wherein said GnRH immunogen in said first and second vaccine compositions is a GnRH multimer comprising the general formula (GnRH-X-GnRH)y wherein:
GnRH is a GnRH immunogen;
X is one or more molecules selected from the group consisting of a peptide linkage, an amino acid spacer group, a carrier molecule and [GnRH]ₙ, where n is an integer greater than or equal to 1; and
y is an integer greater than or equal to 1.

22. The method of claim 20, wherein the carrier molecule is a leukotoxin polypeptide.

23. The method of claim 21, wherein the carrier molecule is a leukotoxin polypeptide.

24. The method of claim 16, wherein the second vaccine composition is administered at about 4 to about 6 weeks before slaughter of the animal.

25. A method of raising an uncastrated male bovine, ovine or porcine animal for meat production comprising:
(a) vaccinating said animal with a first vaccine composition comprising an immunological adjuvant and a GnRH multimer comprising the general formula (GnRH-X-GnRH)y wherein:
GnRH is a GnRH immunogen;
X is one or more molecules selected from the group consisting of a peptide linkage, an amino acid spacer group, a leukotoxin polypeptide and [GnRH]ₙ, where n is an integer greater than or equal to 1; and
y is an integer greater than or equal to 1, wherein said first vaccine composition is administered prior to or during the fattening period of said animal to cause a reduction in circulating testosterone levels; and
(b) vaccinating said animal with a second vaccine composition comprising an immunological adjuvant and a GnRH multimer comprising the general formula (GnRH-X-GnRH)y wherein:
GnRH is a GnRH immunogen;
X is one or more molecules selected from the group consisting of a peptide linkage, an amino acid spacer group, a leukotoxin polypeptide and [GnRH]ₙ, where n is an integer greater than or equal to 1; and
y is an integer greater than or equal to 1, wherein said second vaccine composition is administered at about 2 to about 8 weeks before slaughter of the animal, to substantially reduce the level of one or more androgenic and/or non-androgenic steroids.

26. The method of claim 25, wherein said first and second vaccine compositions comprise the same GnRH multimer.

27. The method of claim 25, wherein said GnRH multimer in the first vaccine composition comprises the amino acid sequence depicted in FIGS. 3A-3F (SEQ ID NO:______), or an amino acid sequence with at least about 75% sequence identity thereto.

28. The method of claim 27, wherein said GnRH multimer comprises the amino acid sequence depicted in FIGS. 3A-3F (SEQ ID NO:______).

29. The method of claim 26, wherein said GnRH multimer in the first and second vaccine compositions comprises the amino acid sequence depicted in FIGS. 3A-3F (SEQ ID NO:______), or an amino acid sequence with at least about 75% sequence identity thereto.

30. The method of claim 29, wherein said GnRH multimer comprises the amino acid sequence depicted in FIGS. 3A-3F (SEQ ID NO:______).

31. The method of claim 25, wherein the second vaccine composition is administered at about 4 to about 6 weeks before slaughter of the animal.

32. A method of raising an uncastrated male bovine, ovine or porcine animal for meat production comprising:
(a) vaccinating said animal with a first vaccine composition comprising an immunological adjuvant and a GnRH multimer comprising the amino acid sequence depicted in FIGS. 3A-3F (SEQ ID NO:______), or an amino acid sequence with at least about 75% sequence identity thereto, wherein said first vaccine composition is administered prior to or during the fattening period of said animal to cause a reduction in circulating testosterone levels; and
(b) vaccinating said animal with a second vaccine composition comprising an immunological adjuvant and a GnRH multimer comprising the amino acid sequence depicted in FIGS. 3A-3F (SEQ ID NO:______), or an amino acid sequence with at least about 75% sequence identity thereto, wherein said second vaccine composition is administered at about 2 to about 8 weeks before slaughter of the animal, to substantially reduce the level of one or more androgenic and/or non-androgenic steroids.

33. The method of claim 32, wherein said GnRH multimer in said first and second vaccine composition comprises the amino acid sequence depicted in FIGS. 3A-3F (SEQ ID NO:______).

34. The method of claim 32, wherein the second vaccine composition is administered at about 4 to about 6 weeks before slaughter of the animal.

35. The method of claim 32, wherein the immunological adjuvant in said first vaccine composition comprises a light mineral oil and dimethyldioctadecylammonium bromide.

35. The method of claim 31, wherein the immunological adjuvant in said second vaccine composition comprises a light mineral oil and dimethyldioctadecylammonium bromide.

36. The method of claim 31, wherein the immunological adjuvant in said first and second vaccine compositions comprises a light mineral oil and dimethyldioctadecylammonium bromide.
